SEQUENCE LISTING (21 sequences)

SEQ ID NO: 1 - protein, 15 aa, Artificial Sequence
  synthetic construct
GGGGSGGGGSGGGGS

SEQ ID NO: 2 - protein, 5 aa, Artificial Sequence
  synthetic construct
GGGGS

SEQ ID NO: 3 - DNA, 23 bp, Artificial Sequence
  synthetic construct
acgtggatccgccaccatggctc

SEQ ID NO: 4 - DNA, 48 bp, Artificial Sequence
  synthetic construct
gggtctgggtgctggagttgtagttgcagagacagtgaccagagtccc

SEQ ID NO: 5 - DNA, 24 bp, Artificial Sequence
  synthetic construct
actacaactccagcacccagaccc

SEQ ID NO: 6 - DNA, 30 bp, Artificial Sequence
  synthetic construct
agtcgaattctcatcatcttggtggcagag

SEQ ID NO: 7 - DNA, 47 bp, Artificial Sequence
  synthetic construct
gggtctgggtgctggagttgtagtcctcttgatctccaccctggtgc

SEQ ID NO: 8 - DNA, 45 bp, Artificial Sequence
  synthetic construct
ggcggcgggggttctggtggcggcggcagcggcggtggaggatca

SEQ ID NO: 9 - DNA, 321 bp, Mouse
gatatccagatgacacagactacatcctccctgtctgcctctctgggagacagagtcaccatcagttgcagggcaagtcaggatattagcaattatttaaactggtatcagcagaaaccagatggaactgttaaactcctgatctactacacatcaagattacactcaggagtcccatcaaggttcagtggcagtgggtctggaacagattattctctcaccattagcaacctggaacaagaagatattgccacttacttttgccaacagggtaatacgcttccgtggacgttcggtggaggcaccaagctggaaatcaaa

SEQ ID NO: 10 - DNA, 363 bp, Mouse
gatgtacgccttcaggagtcaggacctggcctcgtgaaaccttctcagtctctgtctctcacctgctctgtcactggctactccatcaccaatagttattactggaactggattcggcagtttccaggaaacaaactggaatggatggtctacataggctacgacggtagcaataactacaacccatctctcaaaaatcgaatctccatcactcgtgacacatctaagaaccagtttttcctgaagttgaactctgtgactactgaggacacagctacatattactgtgcaagagctacctactatggtaactacagggggtttgcttactggggccaagggactctggtcactgtctctgca

SEQ ID NO: 11 - protein, 107 aa, Mouse
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 12 - protein, 121 aa, Mouse
DVRLQESGPGLVKPSQSLSLTCSVTGYSITNSYYWNWIRQFPGNKLEWMVYIGYDGSNNYNPSLKNRISITRDTSKNQFFLKLNSVTTEDTATYYCARATYYGNYRGFAYWGQGTLVTVSA

SEQ ID NO: 13 - DNA, 336 bp, Mouse
gatgttgtgatgacccaaactccactctccctgcctgtcagtcttggagatcaagcctccatctcttgcagatctagtcagagtcttgtacacagtaatggaaacacctatttacattggaacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 14 - DNA, 354 bp, Mouse
gaggtgcagcttcaggagtcaggacctagcctcgtgaaaccttctcagactctgtccctcacctgttctgtcactggcgactccatcaccagtggttactggaactggctccggaaattcccagggattaaatttgagtacatggggtacataagttacagtggtagcacttactacaatccatctctcaaaagtcgaatctccatcactcgagacacatccaagaaccagtactacctgcagttgaactctgtgactaatgaggacacagccacatattactgtgcaagatactatggtaacggctatgctttggactactggggtcaaggaacctcagtcaccgtctcctca

SEQ ID NO: 15 - protein, 112 aa, Mouse
DVVMTQTPLSLPVSLGDQASISCRSSQSLVHSNGNTYLHWNLQKPGQSPKLLIYKVSNRFSGVPDRFSGSGSGTDFTLKISRVEAEDLGVYFCSQSTHVPWTFGGGTKLEIK

SEQ ID NO: 16 - protein, 118 aa, Mouse
EVQLQESGPSLVKPSQTLSLTCSVTGDSITSGYWNWLRKFPGIKFEYMGYISYSGSTYYNPSLKSRISITRDTSKNQYYLQLNSVTNEDTATYYCARYYGNGYALDYWGQGTSVTVSS

SEQ ID NO: 17 - DNA, 321 bp, Mouse
cagattgtgctgacccagagcccggcgattatgagcgcgagcccgggcgaaaaagtgaccatgacctgcagcgcgagcagcagcgtgtattatatgtattggtatcagcagaaaccgggcagcagcccgcgcctgctgatttatgataccagcaacctggcgagcggcgtgccggtgcgctttagcggcagcggcagcggcaccagctatagcctgaccattagccgcatggaagcggaagatgcggcgacctattattgccagcagtggagcagctatccgccgattacctttggcgtgggcaccaaactggaactgaaa

SEQ ID NO: 18 - DNA, 342 bp, Mouse
gatgtgcagctgcaggaaagcggcccgggcctggtgaaaccgagccagagccagagcctgacctgcaccgtgaccggctatagcattaccagcgattatgcgtggaactggattcgccagtttccgggcaacaaactggaatggatgggctatatgagctatagcggcagcacccgctataacccgagcctgcgcagccgcattagcattacccgcgataccagcaaaaaccagttttttctgcagctgaaaagcgtgaccaccgaagataccgcgacctatttttgcgcgcgcggctggccgctggcgtattggggccagggcacccaggtgagcgtgagc

SEQ ID NO: 19 - protein, 107 aa, Mouse
QIVLTQSPAIMSASPGEKVTMTCSASSSVYYMYWYQQKPGSSPRLLIYDTSNLASGVPVRFSGSGSGTSYSLTISRMEAEDAATYYCQQWSSYPPITFGVGTKLELK

SEQ ID NO: 20 - protein, 114 aa, Mouse
DVQLQESGPGLVKPSQSQSLTCTVTGYSITSDYAWNWIRQFPGNKLEWMGYMSYSGSTRYNPSLRSRISITRDTSKNQFFLQLKSVTTEDTATYFCARGWPLAYWGQGTQVSVS

SEQ ID NO: 21 - protein, 269 aa, Rabbit
TFAQVLTQTPSPVSAAVGGTVTISCQSSESVYGNSRLAWYQQKPGQSPKLLIYYASTLASGVPSRFKGSGSGTQFTLTISDLECDDAASYYCQGGYYSGNLDALAFGGGTEVVVRPNSASHSGSAPNTSSAPGSQQLEQSGGGAEGGLVKPGGSLELCCKASGFSLSSSYCICWVRQAPGKGLEWIGCIYAGSSGSTYYASWVNGRFTLSRDIDQSTGCLQLNSLTAADTAMYYCARAPYSSGWVLYFNLWGPGTLVIVSSGQPKAPSV

(19) United States
(12) Patent Application Publication
(10) Pub. No.: US 2023/0295277 A1
(43) Pub. Date: Sep. 21, 2023

(54) UNIVERSAL CAR-NK CELL TARGETING VARIOUS EPITOPES OF HIV-1 GP160

(71) Applicant: University of Southern California, Los Angeles, CA (US)

(72) Inventors: Rebecca Lim, Los Angeles, CA (US); Liang Rong, Los Angeles, CA (US); Jianming Xie, Los Angeles, CA (US)

(73) Assignee: University of Southern California, Los Angeles, CA (US)

(21) Appl. No.: 18/010,910

(22) PCT Filed: Jul. 13, 2021
(86) PCT No.: PCT/US2021/041469

§ 371 Date: Dec. 16, 2022

Related U.S. Application Data
(60) Provisional application No. 63/051,229, filed on Jul. 13, 2020.

(51) Int. Cl. C07K 16/10; A61K 35/17; C07K 16/28; C12N 15/63; A61P 31/18

(52) U.S. Cl. CPC C07K 16/1045; A61K 35/17; C07K 16/2896; C12N 15/63; A61P 31/18; C07K 2317/622; C07K 2319/03; C07K 2319/02

(57) ABSTRACT

A universal chimeric antigen receptors (CAR)-modified immune cell is provided, including engineering natural killer (NK) cells and/or T cells which recognizes 2,4-dinitrophenyl (DNP) and can subsequently be redirected to target various epitopes of envelop protein gp160 using DNP-conjugated broad neutralizing antibodies as adaptor molecules. In preferred embodiments, the anti-gp160 antibodies target membrane-distal epitopes. This system can recognize and kill mimic HIV-infected cell lines expressing at least one of subtypes B and C gp160. Presently provided system containing universal CAR-NK cells and (bNAbs) overcome the limitations of conventional anti-HIV CARs, as the latter targets a single epitope of the HIV envelope glycoprotein gp160 and falls short of countering the enormous diversity and mutability of viruses.

### CROSS-REFERENCE TO RELATED APPLICATIONS

This application includes a claim of priority under 35 U.S.C. § 119(e) to U.S. provisional patent application No. 63/051,229, filed Jul. 13, 2020, the entirety of which is hereby incorporated by reference.

### STATEMENT REGARDING FEDERALLY SPONSORED RESEARCH OR DEVELOPMENT

This invention was made with Government support under grant no. AI155117 awarded by the National Institutes of Health. The Government has certain rights in the invention.

### REFERENCE TO SEQUENCE LISTING

The Sequence Listing submitted Jul. 13, 2021 as a text file named “SequenceListing-065715-000101WO00_ST25” created on Jul. 8, 2021 and having a size of 13,679 bytes, is hereby incorporated by reference.

### FIELD OF INVENTION

This invention relates to the combination of chimeric antigen receptor (CAR)-engineered immune cells with a broad neutralizing antibody (bNAb)-based adaptor molecule to afford targeting specificity towards multiple epitopes of a disease-specific marker.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Despite three decades of research, human immunodeficiency virus (HIV) infection has remained largely incurable, with more than 36 million people worldwide currently living with the virus. The limitation of current antiretroviral therapy (ART) is that it can effectively suppress HIV replication but not eradicate infected cells, which become latent HIV reservoirs that can induce viral rebound upon treatment interruptions. Developing a method to eradicate HIV-infected cells is thus of critical importance to finding a universal cure for HIV infection.

A promising approach is to use chimeric antigen receptor (CAR)-engineered T cells and natural killer (NK) cells. CARs are hybrid receptors consisting of an extracellular antigen-recognition domain, typically a single-chain variable fragment (scFv) antibody, and an intracellular signaling domain, such as a fusion of CD3ζ and CD28 or 4-1BB fragments that are responsible for T-cell activation. CAR expression can thus enable T cells and also NK cells to recognize and kill target cells expressing the antigen of interest. This approach has been primarily used to treat cancer and achieved remarkable success; for example, the US FDA has approved the use of anti-CD19 CAR-T cells to treat highly refractory B-cell leukemia and lymphoma. It is known that HIV-infected cells express the viral envelope glycoprotein gp160 (a complex between gp120 and gp41) at the cell surface via viral budding. As such, there has been a growing interest in developing anti-gp160 CAR-modified immune cells to eradicate HIV-infected cells. For example, Ali et al. showed that CD8⁺ T cells modified with anti-gp160 CARs could induce lysis of HIV type 1 (HIV-1) infected cells. Additionally, Zhen et al. reported that hematopoietic stem cells engineered with anti-HIV CARs could differentiate into T cells and NK cells and suppress HIV replication in humanized mice.

However, a unique challenge of targeting HIV-1 is that the virus is enormously diverse and highly mutable. HIV-1 is classified into four genetically distinct groups (M, N, O, and P), and the M group alone, which is the most common, contains at least nine subtypes (A-D, F-H, and J-K). The genetic variation of HIV strains can be up to 35% between subtypes and 20% within the same subtype, leading to the expression of extensively diversified gp160 proteins. Since current anti-HIV CARs are mostly designed based on individual broadly neutralizing antibodies (bNAbs) recognizing a single epitope of HIV-1 gp160, they cannot cover all HIV strains. Bi- and tri-specific anti-HIV CARs have recently been developed, which demonstrate enhanced anti-HIV breadth and potency. However, a combination of two or three epitopes is still not sufficient to cover all HIV variants, as indicated by previous studies on multispecific bNAbs. Furthermore, HIV can rapidly mutate under pressure, which may lead to the selection or generation of escape mutants. Therefore, the ability to further expand the epitope coverage of anti-HIV CARs is highly desirable.

Therefore, it is an objective of the present invention to provide an improved system based on CAR-engineered immune cells and bNAbs so as to target universal or multiple epitopes of a marker molecule in diseases such as HIV.

It is another objective of the present invention to provide methods of preparing the improved system and using it for the treatment of related diseases.

### SUMMARY OF THE INVENTION

A combination is provided for use in a treatment, which include a genetically engineered immune cell expressing a chimeric antigen receptor (CAR) specific for 2,4-dinitrophenyl (DNP), and at least one DNP-modified antibody, wherein the DNP-modified antibody is an antibody having a binding affinity specific for a marker associated with a disease or condition (such as human immunodeficiency virus (HIV) or malignant B cells) and the antibody having been modified with a DNP moiety. In various aspects or implementations, the genetically engineered immune cell expressing the CAR specific for the DNP recognizes the at least one DNP-modified antibody, so that the genetically engineered immune cell targets (or in various instances, induces apoptosis of) a cell expressing the HIV-associated, or malignant B cell-associated, marker to which the DNP-modified antibody has the binding affinity.

Another combination is provided, which includes a polynucleotide encoding a chimeric antigen receptor (CAR) specific for 2,4-dinitrophenyl (DNP), or a vector comprising said polynucleotide, and at least one DNP-modified antibody, wherein the DNP-modified antibody has a binding affinity specific for a marker associated with human immunodeficiency virus (HIV) or malignant B cells. In various aspects or implementations, the polynucleotide or the vector upon introduction into an immune cell produces in a genetically engineered immune cell expressing the CAR specific for DNP, said genetically engineered immune cell recognizes (e.g., binds) the at least one DNP-modified antibody and thereby targets (e.g., induces apoptosis of) a cell expressing the HIV-associated or malignant B cell-associated marker to which the DNP-modified antibody has the binding affinity. In further aspects, the combination further includes one or a quantity of immune cells, such as natural killer (NK) cells, or T cells.

In various aspects of the combinations disclosed herein, the immune cell includes a NK cell, or a quantity of NK cells. In other aspects of the combinations disclosed herein, the immune cell includes a T cell, or a quantity of T cells. In further aspects of the combinations disclosed herein, the immune cell is a mixture of NK cells and T cells.

In various aspects of the combinations, the DNP-modified antibody has a binding affinity specific for an HIV-1 envelop glycoprotein gp160, gp 120, or gp41, for example, a broadly neutralizing HIV-1 antibody (bNAb) such as PG9, PGT145, PG16, 2G12, PGT128, PGT121, 10-1074, VRC01, b12, 3BNC117, 3BNC60, 10E8, 4E10, and 2F5. In other aspects, the DNP-modified antibody has affinity specific for CD19. In various implementations, at least two DNP-modified antibodies are provided in the combination, said at least two DNP-modified antibodies bind or target at least two different epitopes of the marker associated with a disease or condition (e.g., HIV or malignant B cells), or at least two different antigens associated with the disease or condition, or at least two different antigens associated with two or more diseases or conditions.

The DNP-modified antibody can be prepared by reacting an antibody with a molecule containing a DNP moiety (or group) and a functional group for conjugation with an antibody. For example, an N-hydroxysuccinimide ester (or succinimidyl) as the function group to react and conjugate with primary amines on an antibody. A non-limiting example of such a molecule is N-(2,4-dinitrophenyl)-6-aminocaproic acid N-succinimidyl ester, which upon reaction with an antibody, introduces a DNP moiety (e.g., N-(2,4-dinitrophenyl)-6-aminocaproic acid moiety) to the antibody. In various aspects, the DNP-modified antibody contains at least two moieties of DNP per molecule of the antibody.

In various aspects, a chimeric antigen receptor (CAR) specific for DNP is provided, which can be that expressed by the genetically engineered immune cell described herein. The CAR contains (a) a DNP-specific targeting region, which can include a variable light chain (VL) of an anti-DNP antibody, a variable heavy chain (VH) of an anti-DNP antibody, or both, (b) a transmembrane domain, and (c) an intracellular signaling domain.

Exemplary VL polypeptide sequences are shown in SEQ ID NO: 11, 15, or 19. Exemplary VH polypeptide sequences are shown in SEQ ID NO: 12, 16, or 20. In various aspects, the DNP-specific targeting region of the CAR is a scFv having a VL and a VH connected by a peptide linker such as that shown in SEQ ID NO:1. An exemplary transmembrane domain includes CD28TM, and an exemplary intracellular signaling domain includes one or more of NKG2D, 2B4, DAP10, CD28, CD16, and CD3ζ.

A method for inducing apoptosis of HIV-1-infected cells or malignant B cells is provided, which includes contacting the HIV-1-infected cells or the malignant B cells with a genetically engineered immune cell expressing the CAR specific for DNP and the at least one DNP-modified antibody, both provided in a combination described herein.

Another method for inducing apoptosis of HIV-1-infected cells or malignant B cells is provided, which includes inducing expression of a CAR specific for DNP in an immune cell by introducing a polynucleotide or a vector described herein (such as that provided in some combinations disclosed), thereby generating a genetically engineered immune cell expressing the CAR specific for DNP, and contacting the HIV-1-infected cells or the malignant B cells with the genetically engineered immune cell in the presence of the at least one DNP-modified antibody described herein (such as that provided in some combinations disclosed).

In various implementations, the genetically engineered cell is provided in a plurality to be contacted with the HIV-infected cells or the malignant B cells at a number ratio ranging from 25:1 to 1:1, such as about 25:1, 20:1, 15:1, 10:1, 5:1, 3:1, 2:1, or 1:1.

In various implementations, the DNP-modified antibody is present in a concentration of between 2 nM and 50 nM, such as about 2-5 nM, 5-10 nM, 10-15 nM, 15-20 nM, 20-30 nM, 30-40 nM, or 40-50 nM.

In further implementations, the method is for inducing apoptosis of HIV-1-infected cells, especially HIV-1-infected cells expressing subtype B envelop protein, and the method includes contacting the genetically engineered immune cell with the HIV-1-infected cells detected with the expression of subtype B envelop protein in the presence of one or a cocktail of DNP-modified, HIV-specific bNAbs selected from PG9, PGT145, PG16, 2G12, PGT128, 10-1074, and 3BNC117, said bNAbs are each modified with a DNP moiety. In some aspects, the method further includes detecting expression of subtype B envelop protein in the HIV-1-infected cells.

In other implementations, the method is for inducing apoptosis of HIV-1-infected cells, especially HIV-1-infected cells expressing subtype C envelop protein, and the method includes contacting the genetically engineered immune cell with the HIV-1-infected cells detected with the expression of subtype C envelop protein in the presence of one or a cocktail of DNP-modified, HIV-specific bNAbs selected from PG9, 10-1074, 3BNC117, and VRC01, said bNAbs are each modified with a DNP group. In some aspects, the method further includes detecting expression of subtype C envelop protein in the HIV-1-infected cells.

Methods of treating a subject inflicted with HIV are provided, which includes administering to the subject an effective amount of the genetically engineered immune cell and the at least one DNP-modified antibody as described in the combination(s) provided, so as to reduce or eradicate the number of HIV-infected cells in the subject (e.g., a human subject). In various implementations, the genetically engineered immune cell is administered in a plurality to the subject first, before the administration of the DNP-modified antibody.

Other methods of treating a subject inflicted with HIV are provided, which includes inducing expression of a CAR specific for DNP in an immune cell by introducing the polynucleotide or the vector provided in the combination(s) disclosed herein into the immune cell, thereby generating a genetically engineered immune cell expressing the CAR specific for DNP, (this step can be performed ex vivo, or in the body of the subject); and administering the genetically engineered immune cell (if generated ex vivo) and administering the at least one DNP-modified antibody provided in the combination(s) disclosed herein to the subject, so as to reduce or eradicate the number of HIV-infected cells in the subject.

In some implementations, the subject's HIV-inflicted cells express subtype B envelop protein gp160, gp120 or gp41, and the at least one HIV-specific bNAb includes PG9, PGT145, PG16, 2G12, PGT128, 10-1074, 3BNC117, or a combination thereof for administration to the subject.

In some implementations, the subject's HIV-inflicted cells express subtype C envelop protein gp160, gp120 or gp41, and the at least one HIV-specific bNAb includes PG9, 10-1074, 3BNC117, VRC01, or a combination thereof for administration to the subject.

In other implementations, the subject's HIV-inflicted cells express both subtype B and subtype C envelop protein gp160, gp120 or gp41, and the at least one HIV-specific bNAb includes at least one selected from PG9, PGT145, PG16, 2G12, PGT128, 10-1074, and 3BNC117 and at least one selected from PG9, 10-1074, 3BNC117, and VRC01.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1C depict generation and characterization of anti-DNP CAR-NK cells. (FIG. 1A) Schematic representation of the anti-DNP CAR construct. CD28TM: CD28 transmembrane domain. (FIG. 1B) Verification of anti-DNP CAR expression on NK-92MI cells after lentiviral transduction and magnetic cell sorting. CAR-NK cells were stained with an anti-HA-tag antibody and a PE-conjugated secondary antibody and then analyzed by flow cytometry. (FIG. 1C) Verification of the DNP-binding ability of CAR-NK cells by flow cytometry. Cells were stained with DNP-conjugated PE.

FIG. 1D is a schematic illustration of using a universal CAR-NK cell to target a plurality of different HIV-infected cells (different HIV variants). The NK cell is engineered to express an anti-DNP CAR, which can be redirected by multiple DNP-labeled antibodies to target different HIV-1 gp160 epitopes expressed on the surface of different infected cells.

FIG. 2A-2D depict redirection of anti-DNP CAR-NK cells to target subtype B gp160-expressing cells by the DNP-conjugated antibody 2G12. (FIG. 2A) Flow cytometry of the subtype B gp160-expressing HEK293 cell line. Expression of gp160 was verified by staining with the anti-gp160 antibody VRC01, followed by a PE-conjugated anti-human IgG antibody. (FIG. 2B) SDS-PAGE of the DNP-modified and unmodified antibody 2G12, followed by coomassie blue staining and western blot analysis using a goat polyclonal anti-DNP antibody. (FIG. 2C) IFN-γ production by anti-DNP CAR-NK cells in response to gp160-positive or negative target cells, with or without DNP-conjugated antibody 2G12 (10 nM). The concentrations of IFN-γ in the culture supernatant were determined by ELISA. Data are presented as the mean ±SD of triplicate samples. Statistical significance is calculated by two-way ANOVA and Tukey's post-hoc analysis. **** p<0.0001. (FIG. 2D) Identification of an optimal concentration of DNP-conjugated 2G12 for activating CAR-NK cells against subtype B gp160+ cells. The concentrations of IFN-γ in the culture supernatant were determined by ELISA. Data are presented as the mean ±SD of triplicate samples. Statistical significance is calculated by two-way ANOVA and Tukey's post-hoc analysis compared with the no antibody control. ** p<0.01, **** p<0.0001.

FIG. 3A-3D depict epitope locations of anti-gp160 antibodies affect their abilities to redirect anti-DNP CAR-NK cells against subtype B gp160⁺ cells. (FIG. 3A) Illustration of the epitope locations of 12 anti-gp160 antibodies tested in this study. The image was generated based on the structures of HIV-1 BG505 SOSIP.664 Env trimer (PDB ID: 5T3Z) and MPER region (PDB ID: 6E8W). (FIG. 3B) IFN-γ production by anti-DNP CAR-NK cells against subtype B gp160-expressing cells in the presence of different DNP-conjugated bNAbs (2 nM). IFN-γ concentrations were determined by ELISA. Data are presented as mean ±SD. Statistical significance is calculated by one-way ANOVA and Dunnett's post-hoc test compared with the isotype control. * p<0.05, ** p<0.01, **** p<0.0001. (FIG. 3C) Cytotoxicity of anti-DNP CAR-NK cells against subtype B gp160⁺ cells at multiple E:T ratios and with different DNP-conjugated bNAbs (2 nM). Cells were stained with a viability dye and analyzed by flow cytometry. The percentage of cytotoxicity was calculated as [(A-B)/Ax100], in which A and B were the numbers of viable gp160⁺ cells after the cell co-culture was incubated with DNP-conjugated isotype control and bNAb, respectively. Data are presented as the mean ±SD of triplicate samples. Statistical significance is calculated by two-way ANOVA and Tukey's post-hoc analysis. * p<0.05, ** p<0.01, **** p<0.0001 vs. the isotype control. ^{##} p<0.01 comparing 3BNC117 to VRC01 at the 25:1 E:T ratio. (FIG. 3D) Analysis of the binding potencies of bNAbs against subtype B gp160⁺ cells. Cells were stained by bNAbs or the isotype control at a series of three-fold dilutions (from 0.045 nM to 300 nM) followed by a PE-conjugated anti-human IgG. The average fluorescence intensity per cell was measured by flow cytometry. Data are presented as the mean ±SD of triplicate samples. Nonlinear regression is used to fit saturation binding curves.

FIGS. 3E and 3F depict universal CAR-NK cell-mediated killing of subtypes B and C gp160⁺ target cells. FIG. 3E depicts the subtype B gp160⁺ HEK293 cells were labelled with dye (carboxyfluorescein succinimidyl ester), while the gp160⁻ HEK293 cells were unlabelled. FIG. 3F depicts the subtype C gp160⁺ HEK293 cells were labelled with carboxyfluorescein succinimidyl ester dye, while the gp160⁻ HEK293 cells were unlabelled. Anti-DNP CAR-NK cells were co-cultured with a 1:1 mixture of gp160⁺ and gp160⁻ cells at different E:T ratios (25:1, 5:1, and 1:1), with or without DNP-conjugated antibodies (2 nM). After an eight-hour incubation, cells were stained with an APC-conjugated anti-human HLA-A2 antibody and the aqua live/dead cell stain reagent, and then subjected to flow cytometric analysis. The gp160⁺ and gp160⁻ HEK293 cells were gated as HLA-A2-positive. Live HEK293 cells after co-culture with CAR-NK cells at the 25:1 E:T ratio are shown above. The percentages in red represent the remaining live gp160⁺ cells compared with the no antibody control (none).

FIG. 4A-4C depict targeting subtype C gp160⁺ cells by universal CAR-NK cells. (FIG. 4A) IFN-γ production by anti-DNP CAR-NK cells against subtype C gp160⁺ cells in the presence of DNP-conjugated bNAbs (2 nM). The concentrations of IFN-γ were determined by ELISA. Data are presented as the mean ±SD of triplicate samples. Statistical significance is calculated by one-way ANOVA and Dunnett's post-hoc test compared with the isotype control. ns: not significant, ** p<0.01, **** p<0.0001. (FIG. 4B) Cytotoxicity of anti-DNP CAR-NK cells against subtype C gp160⁺ cells at multiple E:T ratios and with different DNP-conjugated bNAbs (2 nM). Data are presented as the mean ±SD of triplicate samples. Statistical significance is calculated by two-way ANOVA and Tukey's post-hoc analysis. * p<0.05, **** p<0.0001 vs. the isotype control. ^{###} p<0.001, ^{####} p<0.0001 comparing % cytotoxicity of 10-1074 to VRC01 and 2G12, respectively, at the 25:1 E:T ratio. (FIG. 4C) Flow cytometric analysis of the binding potency of each bNAb against subtype C gp160-expressing cells. Data are presented as the mean ±SD of triplicate samples.

FIG. 5A depicts comparison of the universal and the conventional 2G12-based CAR-NK cells in killing subtype B gp160-expressing cells. Cells were co-cultured eight hours at 1:1, 5:1, or 25:1 E:T ratios. The cytotoxicity was determined based on the percentage of gp160-positive cells killed by CAR-NK cells. Data are presented as the mean ±SD of triplicate samples. Statistical significance is calculated by two-way ANOVA and Tukey's post-hoc analysis. **** p<0.0001 vs. non-transduced NK cells. ## p<0.01 comparing the percentages of cytotoxicity of 2G12-CAR-NK cells to that of DNP-CAR-NK cells at the 25:1 E:T ratio.

FIG. 5B depicts that universal CAR-NK cells mediate an effective killing of HIV-infected CD4+ T cells. CAR-NK cells and HIV-infected CD4+ T cells were cocultured at a 20:1 E/T ratio with or without DNP-modified bNAbs (10 nM). After overnight incubation, the cytotoxicity of CAR-NK cells was assessed by flow cytometry. % killing was calculated as [(A−B)/A×100], in which A and B were the percentages of live Gag-expressing CD4+ T cells without and with the treatment of CAR-NK cells, respectively. Data are presented as the mean ±SD of duplicate samples. Statistical significance is calculated by a one-way ANOVA and Tukey's post hoc analysis. *p<0.05, **p<0.01, vs the negative control in which no antibody was added.

FIG. 6 depicts that the unconjugated antibody PG9 cannot redirect anti-DNP CAR-NK cells to target gp160⁺ cells. Anti-DNP CAR-NK cells were co-cultured with the subtype B gp160-expressing cells in the presence of DNP-conjugated and unconjugated antibody PG9. The concentrations of IFN-γ in the culture supernatant were determined by ELISA. Data are presented as the mean ±SD of triplicate samples. Statistical significance is calculated by two-way ANOVA and Tukey's post-hoc analysis compared with the unconjugated PG9. **** P<0.0001.

FIG. 7 depicts redirection of the universal CAR-NK cell to target malignant B cells. Anti-DNP CAR-NK cells were co-cultured with REH cells at a 25:1 E:T ratio with or without a DNP-conjugated anti-CD19 antibody (clone FMC63, 50 nM). After overnight incubation, the percentages of remaining REH cells were determined by flow cytometry. CD56 is a cell surface marker for NK cells.

FIG. 8 depicts the targeting of a mixture of subtype B and C gp160-expressing cells by CAR-NK cells supplemented with individual or combined bNAbs. The percentage of CAR-NK cell cytotoxicity was calculated as [(A-B)/Ax100], in which A and B were the numbers of viable gp160⁺ cells (both subtypes B and C) after the cell co-culture was incubated without and with DNP-conjugated antibodies, respectively. Data are presented as the mean ±SD of triplicate samples. Statistical significance is calculated by two-way ANOVA and Tukey's post-hoc analysis. *** p<0.001, **** p<0.0001 vs. the isotype control.

FIG. 9 depicts that universal anti-DNP CAR-T cells can also kill subtype B gp160+ cells. Primary human anti-DNP CAR-T cells were co-cultured with CellTrace Blue-labeled HEK293 gp160+ Subtype B cells at a 1:1 E:T ratio in the presence of 10 nM of DNP-conjugated bNAbs or isotype control. After a 48-hour incubation at 37° C., cells were collected and stained with a FITC-conjugated anti-human CD3 antibody and an aqua live/dead cell stain reagent, followed by flow cytometric analysis. The percentage of CAR-T cell cytotoxicity was calculated as [(A-B)/Ax100], in which A and B were the numbers of viable gp160⁺ cells after the cell co-culture was incubated without and with the addition of DNP-conjugated antibodies, respectively. Data are presented as the mean ±SD of triplicate samples. Statistical significance is calculated by one-way ANOVA and Dunnett's post-hoc analysis compared to the isotype control. ** p<0.01, *** p<0.001. n.s.: not significant.

FIG. 10 depicts comparison of the distances of different gp160 and CD22 epitopes to the cell membrane. The extracellular domain of CD22 consists of seven Ig-like domains. Based on the structure of CD22 (PDB ID: 5VKJ), the average length of each Ig-like domain is estimated to be about 38 Å.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. Sep; 23(9):1126-36).

One skilled in the art will recognize many methods and materials similar or equivalent to those described herein, which could be used in the practice of the present invention. Indeed, the present invention is in no way limited to the methods and materials described.

“Chimeric antigen receptor” or “CAR” or “CARs” refers to engineered receptors, which graft an antigen specificity onto cells (for example natural killer (NK) cells, T cells such as naïve T cells, central memory T cells, effector memory T cells or combination thereof).

“Transmembrane domain” (TMD) refers to a region of the CAR which crosses the plasma membrane. For example, the transmembrane domain of a CAR can be the transmembrane region of a transmembrane protein (for example Type I transmembrane proteins), an artificial hydrophobic sequence or a combination thereof. Other transmembrane domains will be apparent to those of skill in the art and may be used in connection with alternate embodiments of the invention.

“Intracellular signaling domain” (ISD) or “cytoplasmic domain” or “signaling domain” refers to the portion of the CAR which transduces the effector function signal and directs the cell to perform its specialized function. Examples of domains that transduce the effector function signal include but are not limited to the ζ chain of the T-cell receptor complex or any of its homologs (e.g., η chain, FcεR1γ and β chains, MB1 (Igα) chain, B29 (Igβ) chain, etc.), human CD3 zeta chain, CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. Other intracellular signaling domains will be apparent to those of skill in the art and may be used in connection with alternate embodiments of the invention.

“Extracellular spacer domain” (ESD) refers to a generally hydrophilic region which is between the antigen-specific targeting region and the transmembrane domain. The extracellular spacer domains include but are not limited to Fc fragments of antibodies or fragments or derivatives thereof, hinge regions of antibodies or fragments or derivatives thereof, CH2 regions of antibodies, CH3 regions of antibodies, artificial spacer sequences or combinations thereof. Examples of extracellular spacer domains include but are not limited to CD8α hinge, and artificial spacers made of polypeptides which may be as small as, for example, Gly3 or CH1 and CH3 domains of IgGs (such as human IgG4). In some embodiments, the extracellular spacer domain is any one or more of (i) a hinge, CH2 and CH3 regions of IgG4, (ii) a hinge region of IgG4, (iii) a hinge and CH2 of IgG4, (iv) a hinge region of CD8α, (v) a hinge, CH2 and CH3 regions of IgG1, (vi) a hinge region of IgG1 or (vi) a hinge and CH2 region of IgG1. Other extracellular spacer domains will be apparent to those of skill in the art and may be used in connection with alternate embodiments of the invention.

“Linker” (L) or “linker domain” or “linker region” refers to an oligo- or polypeptide region from about 1 to 100 amino acids in length, which links together any of the domains/regions of the CAR of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, Thosea asigna virus (T2A) or combinations, variants and functional equivalents thereof. Other linkers will be apparent to those of skill in the art and may be used in connection with alternate embodiments of the invention.

“Co-stimulatory domain” (CSD) refers to the portion of the CAR which enhances the proliferation, survival and/or development of memory cells. The CARs of the invention may comprise one or more co-stimulatory domains. Each co-stimulatory domain comprises the costimulatory domain of any one or more of, for example, members of the TNFR superfamily, CD28, CD137 (4-1BB), CD134 (OX40), Dap10, CD27, CD2, CD5, ICAM-1, LFA-1(CD11a/CD18), Lck, TNFR-I, TNFR-II, Fas, CD30, CD40 or combinations thereof. Other co-stimulatory domains (e.g., from other proteins) will be apparent to those of skill in the art and may be used in connection with alternate embodiments of the invention.

HIV-1 envelope glycoprotein “gp160” is encoded by the HIV-1 env gene, which is subsequently cleaved into the envelope proteins gp120 and gp41. The Env glycoprotein, a complex between gp120 and membrane-bound gp41, is expressed on both the surface of the HIV virus and on virus-infected cells. Without wishing to be bound by a particular theory, the gp120 component of Env mediates the first step in viral entry into human cells by binding the protein CD4. gp120 is the viral surface protein that mediates attachment of the virus to target cells via binding to the CD4 receptor and a co-receptor, most commonly the chemokine receptors CCR5 or CXCR4. gp41 is a transmembrane protein that mediates fusion of the viral and cellular lipid membranes once gp120 has bound its cognate receptor and co-receptor.

“Treat”, “treating”, and “treatment”, etc., refer to any action providing a benefit to a patient. In various aspects, “treat”, “treating”, and “treatment” refer to an action providing a benefit to a patient at risk for HIV infection or having an HIV infection, including improvement in the condition through lessening or suppression of titers of HIV or at least one symptom of HIV, prevention or delay in progression of the disease, prevention or delay in the onset of disease states or conditions which occur secondary to HIV, including AIDS or ARC, among others. Treatment, as used herein, encompasses both prophylactic and therapeutic treatment. The term “prophylactic” when used, means to reduce the likelihood of an occurrence or the severity of an occurrence within the context of the treatment of HIV, as otherwise described hereinabove.

“Human immunodeficiency virus” or “HIV” describes human immunodeficiency viruses 1 and 2 (HIV-1 and HIV-2). Viruses which may be treated according to the present invention include, for example, human immunodeficiency viruses 1 and 2 (HIV-1 and HIV-2), among others. The term HIV includes mutant strains of HIV including “drug resistant” or “multiple drug resistant” strains of the HIV virus which have mutated to be resistant to one or more clinically approved anti-HIV agents, including, in particular, HIV strains which are resistant to one or more NRTI compounds and/or NNRTI compounds. Exemplary HIV drug resistant strains which may be effectively treated using compounds according to the present invention include the following, among others: (defined by their reverse transcriptase or RT mutation)—XXBRU, K65R, Y115F, F116Y, Q151M, M184V, L74V, V75T, 4XZT, T215Y, K103N, T215Y/M184V, 5705-72, 488-101, C910-6, LA1M184V, G910-6 L100I, K101E, K103N, V106A, D110E, V179D, Y181C, D185E, D186E, Y188H, G190E, E138K, M41L, D67N, K70R, T215Y/F, K219Q/E, Y181C, K103N, L100I, Y188C/H, among others, including HIV-1 isolates JR-FL, ADA, HXBc2, SF162 and BaL, among others.

“ARC” and “AIDS” refer to syndromes of the immune system caused by the human immunodeficiency virus, which are characterized by susceptibility to certain diseases and T cell counts which are depressed compared to normal counts. HIV progresses from Category 1 (Asymptomatic HIV Disease) to Category 2 (ARC), to Category 3 (AIDS), with the severity of the disease.

“B-cell associated diseases” or “Malignant B-cell” associated diseases or conditions, suitable for treatment by use of one or more methods disclosed herein, include B-cell immunodeficiencies, autoimmune diseases and/or excessive/uncontrolled cell proliferation associated with B-cells (including lymphomas and/or leukemias). Examples of such diseases, wherein bispecific CARs of the invention may be used for therapeutic approaches include but are not limited to systemic lupus erythematosus (SLE), diabetes, rheumatoid arthritis (RA), reactive arthritis, multiple sclerosis (MS), pemphigus vulgaris, celiac disease, Crohn's disease, inflammatory bowel disease, ulcerative colitis, autoimmune thyroid disease, X-linked agammaglobulinaemis, pre-B acute lymphoblastic leukemia, systemic lupus erythematosus, common variable immunodeficiency, chronic lymphocytic leukemia, diseases associated with selective IgA deficiency and/or IgG subclass deficiency, B lineage lymphomas (Hodgkin's lymphoma and/or non-Hodgkin's lymphoma), immunodeficiency with thymoma, transient hypogammaglobulinaemia and/or hyper IgM syndrome, as well as virally-mediated B-cell diseases such as EBV mediated lymphoproliferative disease, and chronic infections in which B-cells participate in the pathophysiology.

A “neutralizing antibody” (NAb) refers to an antibody that defends a cell from a pathogen or infectious particle by neutralizing any effect it has biologically. HIV-specific broadly neutralizing antibodies (bNAbs) are neutralizing antibodies which neutralize multiple HIV-1 viral strains. bNAbs are unique in that they target conserved epitopes of the virus, meaning the virus may mutate, but the targeted epitopes will still exist.

“Coadministration” or “combination therapy” refers to at least two compounds or compositions are administered to the patient at the same time, or at different times, such that effective amounts or concentrations of each of the two or more compounds may be found in the patient at a given point in time. In certain preferred aspects, one or more engineered immune cells expressing a chimeric antigen receptor with a DNP-specific targeting region are coadministered in combination with at least one DNP-modified anti-gp160 antibody (including DNP-modified anti-gp120 antibody and/or DNP-modified anti-gp41 antibody) or at least one DNP-modified, HIV-specific bNAb in a cocktail for the treatment of HIV infections. In certain aspects, one or more immune cells are genetically engineered to express a chimeric antigen receptor with a DNP-specific targeting region, said genetically engineered immune cells preferably being autologous or allogeneic NK cells or T cells, and at least one DNP-modified, HIV-specific bNAb, or at least one DNP-modified anti-gp160 antibody (including DNP-modified anti-gp120 antibody, and/or DNP-modified anti-gp41 antibody), is administered to a subject containing such immune cells.

Described herein includes engineered natural killer (NK) cells and T cells with anti-HIV chimeric antigen receptors (CAR) as a promising strategy to eradicate HIV-infected cells. Current anti-HIV CARs are limited by targeting a single epitope of the HIV envelope glycoprotein gp160, which cannot counter the enormous diversity and mutability of viruses. Here, we report the development of a universal CAR-NK cell, which recognizes 2,4-dinitrophenyl (DNP) and can subsequently be redirected to target various epitopes of gp160 using DNP-conjugated antibodies as adaptor molecules. Several DNP-conjugated antibodies are prepared based on PG9, PGT145, PG16, 2G12, PGT128, 10-1074, VRC01, b12, 3BNC117, 10E8, 4E10, 2F5, and FMC63, wherein each of these antibodies are modified with at least one molecule of DNP, e.g., these antibodies having a DNP group or a N-(2,4-dinitrophenyl)-6-aminocaproic acid moiety. We show that this CAR-NK cell can recognize and kill mimic HIV-infected cell lines expressing subtypes B and C gp160. We additionally found that anti-gp160 antibodies targeting membrane-distal epitopes (including V1/V2, V3, and CD4bs) are more likely to activate universal CAR-NK cells against gp160⁺ cells, compared with those targeting membrane-proximal epitopes located in the gp41 MPER. Finally, we confirm that HIV-infected primary human CD4+ T cells can be effectively killed using the same approach. Given that numerous anti-gp160 antibodies with different specificities are available, this approach can significantly expand the epitope coverage of anti-HIV CAR-NK cells, thus providing a promising strategy to overcome viral diversity.

Our universal CAR-NK cell approach presents several improvement and advantages over conventional antibody-dependent cellular cytotoxicity (ADCC). First, the universal CAR is more versatile because it is compatible with all types of antibodies, including IgA which cannot effectively induce NK cell-mediated ADCC. Second, it is likely more specific and safer, because anti-DNP CAR-NK cell-mediated killing is strictly dependent on DNP-conjugated antibodies and thus will not be induced by serum antibodies. Third, the potency of universal CAR-NK cells can be further enhanced by incorporating multiple signaling domains. In some embodiments, the anti-DNP CAR contains a transmembrane domain and an intracellular signaling domain of NKG2D, 2B4, DAP10, and CD3ζ, allowing for more effectively killing of ovarian cancer cells, as compared to a CAR containing the CD16 intracellular signaling domain. Finally, the universal CAR-NK cell approach and ADCC are not mutually exclusive because the DNP-conjugated bNAbs can still recruit primary NK cells to respond to gp160⁺ cells.

### Anti-DNP Chimeric Antigen Receptors, Polynucleotides, and Engineered Immune Cells

Various embodiments provide a chimeric antigen receptor, comprising (a) an antigen-specific targeting region, which is specific to the 2,4-dinitrophenyl group (DNP), recognizing and/or binding the DNP epitope, (also called DNP-specific targeting region,) (b) a transmembrane domain, and (c) an intracellular signaling domain.

An antigen-specific targeting region, in various implementations, may comprise single chain variable fragment (scFv), full length heavy chain, Fab fragments, divalent single chain antibodies or diabodies, each of which are specific to the target antigen, DNP. In fact, a molecule that binds DNP with high affinity can be used as an antigen-specific targeting region, as will be appreciated by those of skill in the art.

An exemplary DNP-specific targeting region comprises a polypeptide sequence comprising a variable heavy chain (V_{H}) specific for DNP, a variable light chain (V_{L}) specific for DNP, or both a V_{H} specific for DNP and a V_{L} specific for DNP, connected by a peptide linker (e.g., a peptide linker of SEQ ID NO:1, or a peptide linker having one, two, four, five, six, or more repeating units of SEQ ID NO:2). In some aspects, the DNP-specific targeting region of a chimeric antigen receptor of the invention comprises a scFv, e.g., in the form of V_{L}-linker-V_{H} from N- to C-terminus. In other aspects, the DNP-specific targeting region of a chimeric antigen receptor of the invention comprises a scFv in the form of V_{H}-linker-V_{L} from N- to C-terminus. Yet in further aspects, the DNP-specific targeting region of a chimeric antigen receptor of the invention comprises a single-domain antibody.

An exemplary DNP-specific targeting region of a chimeric antigen receptor, with high affinity towards DNP, include a variable light chain polypeptide sequence of SEQ ID NO:11; a variable heavy chain polypeptide sequence of SEQ ID NO:12; or both SEQ ID NO:11 and SEQ ID NO:12, connected by a peptide linker (e.g., linker sequence SEQ ID NO:1).

Another exemplary DNP-specific targeting region of a chimeric antigen receptor include a variable light chain polypeptide sequence of SEQ ID NO:15; a variable heavy chain polypeptide sequence of SEQ ID NO:16; or both SEQ ID NO:15 and SEQ ID NO:16, connected by a peptide linker (e.g., linker sequence SEQ ID NO:1).

Another exemplary DNP-specific targeting region of a chimeric antigen receptor include a variable light chain polypeptide sequence of SEQ ID NO:19; a variable heavy chain polypeptide sequence of SEQ ID NO:20; or both SEQ ID NO:19 and SEQ ID NO:20, connected by a peptide linker (e.g., linker sequence SEQ ID NO:1).

In some aspects, a DNP-specific targeting region of a chimeric antigen receptor of the invention comprises V_{H} and V_{L} of an anti-DNP antibody, connected by a peptide linker (e.g., a peptide linker of SEQ ID NO:1, or a peptide linker having one, two, four, five, six, or more repeating units of SEQ ID NO:2). In some aspects, a DNP-specific targeting region of a chimeric antigen receptor of the invention comprises V_{H} of an anti-DNP antibody. In some aspects, a DNP-specific targeting region of a chimeric antigen receptor of the invention comprises V_{L} of an anti-DNP antibody.

An anti-DNP antibody can be IgG1, IgG2a, IgG3, IgA type, or another type. Various anti-dinitrophenyl monoclonal antibodies, as well as polyclonal antibodies, are available and described in the art, including Leahy et al., Proc. Natl. Acad. Sci. USA, 1988, 85:3661-3665; Bassolino-Klimas et al., Protein Science, 1992, 1, 1465-1476; Gonzalez et al., J Immunol, 2000, 164:1071-1077, which are incorporated by reference. Additional exemplary anti-DNP antibodies are described in WO2009111729, which is incorporated by reference, including an exemplary rabbit anti-DNP scFv having a polypeptide sequence of SEQ ID NO:21.

In various embodiments, a chimeric antigen receptor includes a transmembrane domain, CD28TM, and an intracellular signaling domain containing CD28 and CD3ζ. CD28 may also be referred to as a co-stimulatory domain.

In some embodiments, the chimeric antigen receptors described herein may be synthesized as single polypeptide chains and may comprise at least one DNP-specific targeting region (or two or more DNP-specific targeting regions), a transmembrane domain, and an intracellular signaling domain. In this embodiment, the DNP-specific targeting region(s) are at the N-terminus, arranged in tandem if more than one DNP-specific targeting region is used. The antigen-specific targeting region is linked to the transmembrane domain, and the transmembrane domain is linked on another end to the intracellular signaling domain which is at the C-terminus. The antigen-specific targeting region may be extracellular-facing and the intracellular signaling domain may be cytoplasmic. In one embodiment, an anti-DNP chimeric antigen receptor may be in the following configuration from the N-terminal to C-terminal direction: N-terminal signal sequence—DNP-specific targeting region—transmembrane domain—intracellular signaling domain. In one embodiment, an anti-DNP chimeric antigen receptor may be in the following configuration from the N-terminal to C-terminal direction: DNP-specific V_{L}—linker—DNP-specific V_{H}—transmembrane domain—intracellular signaling domain. In another embodiment, an anti-DNP chimeric antigen receptor may be in the following configuration from the N-terminal to C-terminal direction: DNP-specific V_{H}—linker—DNP-specific V_{L}—transmembrane domain—intracellular signaling domain.

Further embodiments provide that a chimeric antigen receptor further comprises one or more of (d) an extracellular spacer domain, positioned between the DNP-specific targeting region and the transmembrane domain, and (e) one or more co-stimulatory domains, positioned between the transmembrane domain and the intracellular signaling domain. In other embodiments, a chimeric antigen receptor specific for DNP does not comprise an extracellular spacer domain, a co-stimulatory domain, or both.

A polynucleotide encoding a DNP-specific chimeric antigen receptor is also provided, wherein the polynucleotide comprises a sequence encoding a DNP-specific targeting region containing V_{L}, V_{H}, and a linker positioned in between, said sequence comprising SEQ ID NO:9 (which encodes a DNP-specific V_{L}), SEQ ID NO:10 (which encodes a DNP-specific V_{H}), and SEQ ID NO:8 (which encodes a linker). In some aspects, the polynucleotide comprising a sequence encoding a DNP-specific targeting region contains SEQ ID NO:9 (which encodes a DNP-specific V_{L}), with no sequence encoding V_{H}. In some aspects, the polynucleotide comprising a sequence encoding a DNP-specific targeting region contains SEQ ID NO:10 (which encodes a DNP-specific V_{H}), with no sequence encoding V_{L}.

Another polynucleotide encoding a DNP-specific chimeric antigen receptor is provided, wherein the polynucleotide comprises a sequence encoding a DNP-specific targeting region containing V_{L}, V_{H}, and a linker positioned in between, said sequence comprising SEQ ID NO:13 (which encodes a DNP-specific V_{L}), SEQ ID NO:14 (which encodes a DNP-specific V_{H}), and SEQ ID NO:8 (which encodes a linker). In some aspects, the polynucleotide comprising a sequence encoding a DNP-specific targeting region contains SEQ ID NO:13 (which encodes a DNP-specific V_{L}), with no sequence encoding V_{H}. In some aspects, the polynucleotide comprising a sequence encoding a DNP-specific targeting region contains SEQ ID NO:14 (which encodes a DNP-specific V_{H}), with no sequence encoding V_{L}.

Yet an additional polynucleotide encoding a DNP-specific chimeric antigen receptor is also provided, wherein the polynucleotide comprises a sequence encoding a DNP-specific targeting region containing V_{L}, V_{H}, and a linker positioned in between, said sequence comprising SEQ ID NO:17 (which encodes a DNP-specific V_{L}), SEQ ID NO:18 (which encodes a DNP-specific V_{H}), and SEQ ID NO:8 (which encodes a linker). In some aspects, the polynucleotide comprising a sequence encoding a DNP-specific targeting region contains SEQ ID NO:17 (which encodes a DNP-specific V_{L}), with no sequence encoding V_{H}. In some aspects, the polynucleotide comprising a sequence encoding a DNP-specific targeting region contains SEQ ID NO:18 (which encodes a DNP-specific V_{H}), with no sequence encoding V_{L}.

In various embodiments, a polynucleotide encoding a DNP-specific chimeric antigen receptor comprises a sequence encoding a DNP-specific targeting region, a sequence encoding a transmembrane domain, and a sequence encoding an intracellular signaling domain.

A vector is also provided, which encodes a DNP-specific targeting region or an anti-DNP chimeric antigen receptor, comprising a polypeptide sequence of any one or more of SEQ ID NOs: 9, 10, 13, 14, 17, and 18 and SEQ ID NO:8. In some aspects, a vector encoding an anti-DNP chimeric antigen receptor comprises an expression cassette of a polynucleotide sequence of SEQ ID NO:9, SEQ ID NO:10, or a combination of SEQ ID NOs:9, 8 and 10. In other aspects, a vector encoding an anti-DNP chimeric antigen receptor comprises an expression cassette of a polynucleotide sequence of SEQ ID NO:13, SEQ ID NO:14, or a combination of SEQ ID NOs:13, 8 and 14. In further aspects, a vector encoding an anti-DNP chimeric antigen receptor comprises an expression cassette of a polynucleotide sequence of SEQ ID NO:17, SEQ ID NO:18, or a combination of SEQ ID NOs:17, 8 and 18.

Vectors which may be used to express the chimeric antigen receptor of the invention include but are not limited to lentivirus vectors, gamma retrovirus vectors, foamy virus vectors, AAV vectors, adeno virus vectors, engineered hybrid viruses, naked DNA (including but not limited to transposon mediated vectors, such as Sleeping Beauty, Piggybak, and Integrases such as Phi31. In an exemplary embodiment, an anti-DNP chimeric antigen receptor disclosed herein is expressed via a lentiviral vector.

Genetically engineered cells are also provided which comprise and stably express a DNP-specific chimeric antigen receptor disclosed herein. In various embodiments, genetically engineered cells are introduced with one or more polynucleotides encoding a DNP-specific chimeric antigen receptor. The chimeric antigen receptor expressed by the genetically engineered cell may comprise at least one DNP-specific targeting region, a transmembrane domain, and an intracellular signaling domain. The polynucleotide sequence encoding the chimeric antigen receptor may also comprise an N-terminal signal sequence. Each of the DNP-specific targeting region, transmembrane domain, and an intracellular signaling domain, and if applicable extracellular spacer domain and one or more co-stimulatory domains, are described above.

In some embodiments, the DNP-specific targeting region of the chimeric antigen receptor expressed by the genetically engineered cell(s) is a Fab fragment of an anti-DNP antibody or a scFv of an anti-DNP antibody.

Genetically engineered cells which comprise and express the DNP-specific chimeric antigen receptors of the invention include, but are not limited to, natural killer cells, T-lymphocytes (T-cells), naïve T cells (T_{N}), memory T cells (for example, central memory T cells (T_{CM}), effector memory cells (T_{EM})), hematopoietic stem cells and/or pluripotent embryonic/induced stem cells capable of giving rise to therapeutically relevant progeny. In an embodiment, the genetically engineered cells are autologous cells.

In some embodiments the immune cells that express the engineered CAR are NK cells; and in further embodiments, the immune cells are allogeneic NK cells. In some embodiments, the immune cells that express the engineered CAR are T cells.

In an exemplary embodiment, the genetically engineered natural killer cells of the invention express a DNP-specific chimeric antigen receptor, wherein the DNP-specific chimeric antigen receptor comprises a DNP-specific targeting region, a transmembrane domain, and an intracellular domain, and the DNP-specific targeting region may be a scFv including a polypeptide variable light chain of SEQ ID NO: 11, 15, or 19, a polypeptide variable heavy chain of SEQ ID NO: 12, 16, or 20, or a combination of a polypeptide variable light chain of SEQ ID NO:11 and a polypeptide variable heavy chain of SEQ ID NO:12, a combination of a polypeptide variable light chain of SEQ ID NO:15 and a polypeptide variable heavy chain of SEQ ID NO:16, or a combination of a polypeptide variable light chain of SEQ ID NO:19 and a polypeptide variable heavy chain of SEQ ID NO:20, optionally further comprising a linker of SEQ ID NO:1.

Genetically modified cells may be produced by stably transfecting cells with DNA encoding the DNP-specific chimeric antigen receptor of the invention. Viral vectors are commonly used to carry heterologous genes into cells (e.g., NK cells, or T-cells). Examples of viral vectors which may be used to generate genetically modified cells include but are not limited to SIN lentiviral vectors, retroviral vectors, foamy virus vectors, adeno-associated virus (AAV) vectors and/or plasmid transposons (e.g., sleeping beauty transposon system).

Various methods produce stable transfectants which express the DNP-sepcific chimeric antigen receptor of the invention. In one embodiment, a method of stably transfecting and re-directing cells is by electroporation using naked DNA. By using naked DNA, the time required to produce redirected cells may be significantly reduced. Additional methods to genetically engineer cells using naked DNA encoding the DNP-specific chimeric antigen receptor include but are not limited to chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). The transfected cells demonstrating presence of a single integrated un-rearranged vector and expression of the DNP-specific chimeric antigen receptor may be expanded ex vivo.

### Systems or Combinations

Various embodiments provide a combination, or a system, including (1) a genetically engineered cell expressing a DNP-specific chimeric antigen receptor or introduced with a polynucleotide encoding a DNP-specific chimeric antigen receptor, and (2) at least one DNP-modified antibody having a binding affinity specific for an antigen or marker associated with a disease of condition. In various aspects, the DNP-modified antibody is a broadly neutralizing antibody (bNAb), wherein the at least one bNAb is modified with a DNP group. In some aspects, the at least one bNAb modified with a DNP group in the combination is HIV-specific bNAb, and the genetically engineered cell is a NK cell or a T cell. In other aspects, the at least one bNAb modified with a DNP group in the combination is a bNAb specific for or targeting another marker associated with a disease or condition, e.g., CD19, a biomarker overexpressed in most B-cell malignancies.

In various embodiments, the combination includes a plurality of genetically engineered cells expressing a DNP-specific chimeric antigen receptor and 2, 3, 4, 5, 6, 7, 8, 9, 10, 11, 12 or more HIV-specific, DNP-modified bNAbs, each bNAbs targeting a different epitope of gp160 (or gp120 and gp41) of a same subtype or different subtypes of HIV-1. In some aspects, the combination includes a plurality of the genetically engineered cells expressing a DNP-specific chimeric antigen receptor and two HIV-specific, DNP-modified bNAbs, each targeting a different epitope of HIV-1. In some aspects, the combination includes a plurality of the genetically engineered cells expressing a DNP-specific chimeric antigen receptor and three HIV-specific, DNP-modified bNAbs, each targeting a different epitope of HIV-1. In some aspects, the combination includes a plurality of the genetically engineered cells expressing a DNP-specific chimeric antigen receptor and four HIV-specific, DNP-modified bNAbs, each targeting a different epitope of HIV-1.

Exemplary HIV-1-specific bNAbs include but are not limited to those targeting MPER of gp41, i.e., antibody clonal families 2F5, 4E10, M66.6, CAP206-CH12, and 10E8 I; those targeting V1V2-glycan, i.e., antibody clonal families PG9, PG16, CH01-04, PGT 141-145; those targeting outer domain glycan, e.g., 2G12; those targeting V3-glycan, e.g., PGT121-123, PGT125-131, and PGT135-137; and those targeting CD4 binding site, e.g., b12, HJ16, CH103-106, VRC01-03, VRC-PG04, 04b, VRC-CH30-34, 3BNC117, 3BNC60, NIH45-46, 12A12, 12A21, 8ANC131, 134, 1NC9, and 1B2530.

In an exemplary embodiment, the two or more of HIV-specific bNAbs in the combination or system, coupled or modified with a DNP group, include any two or all three of PG9, PGT145, and PG16. In another exemplary embodiment, the two or more of HIV-specific bNAbs in the combination or system, coupled or modified with a DNP group, include any two or all three of 2G12, PGT128, and 10-1074. In yet another exemplary embodiment, the two or more of HIV-specific bNAbs in the combination or system, coupled or modified with a DNP group, include any two or more of PG9, PGT145, PG16, 2G12, PGT128, 10-1074, and 3BNC117. In another exemplary embodiment, the two or more of HIV-specific bNAbs in the combination or system, coupled or modified with a DNP group, do not include VRC01, b12, 10E8, 4E10 or 2F5. These combinations or systems can be used in inducing apoptosis of HIV-infected cells expressing subtype B gp160.

In yet another exemplary embodiment, the HIV-specific bNAb(s) in the combination or system, coupled or modified with a DNP group, include any one, two, three, or all four of PG9, 10-1074, 3BNC117, and VRC01, but do not include 2G12 or 10E8; and this combination or system can be used in inducing apoptosis of HIV-infected cells expressing subtype C gp160.

Antigens associated with a cancer, and to which an antibody has affinity, include but are not limited to any one or more of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2 or vimentin.

Antigens associated with an inflammatory disease, and to which an antibody has affinity, include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, Lama glama, LFA-1 (CD11a), MEDI-528, myostatin, OX-40, rhuMAb β7, scleroscin, SOST, TGF beta 1, TNF-α or VEGF-A.

Antigens associated with a cardiovascular disease, and to which an antibody has affinity, include but are not limited to any one or more of C5, cardiac myosin, CD41 (integrin alpha-IIb), fibrin II, beta chain, ITGB2 (CD18) and sphingosine-1-phosphate.

Antigens associated with an infectious disease, and to which an antibody has affinity, include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α.

Any known antibody in the art with specific affinity to the antigen described herein can be modified with a DNP moiety, and be included in one or more of the combinations or used in the one or more methods described herein.

Various embodiments further provide a combination, or a system, including (1) a polynucleotide encoding a DNP-specific chimeric antigen receptor, or a vector comprising said polynucleotide, and (2) at least one broadly neutralizing antibody (bNAb), wherein the at least one bNAb is modified with a DNP group.

Yet further embodiments provide a system or a combination, including (1) a polynucleotide encoding a DNP-specific chimeric antigen receptor, or a vector comprising said polynucleotide, (2) a natural killer cell, or a T cell, and (3) at least one broadly neutralizing antibody (bNAb) modified with a DNP group.

In various implementations, the polynucleotide encoding a DNP-specific chimeric antigen receptor, or the vector comprising said polynucleotide, is used to induce expression of the DNP-specific chimeric antigen receptor in a natural killer cell or a T cell (or the natural killer cell or the T cell if included in the system), and the at least one bNAb modified with a DNP group is used to direct the natural killer cell (or T cell) expressing the DNP-specific chimeric antigen receptor to recognize and/or induce apoptosis of target cells expressing a biomarker that is recognized by the bNAb.

Further embodiments provide a combination, or a system, including (1) a genetically engineered natural killer cell or T cell, which is introduced with a polynucleotide encoding a DNP-specific chimeric antigen receptor, or expressing the DNP-specific chimeric antigen receptor, and (2) two or more antibodies, each modified with a DNP group, said two or more antibodies bind and/or block two or more antigens associated with a disease or condition, or two or more epitopes of an antigen associated with a disease of condition.

Other embodiments provide a combination, or a system, including (1) polynucleotide encoding a DNP-specific chimeric antigen receptor, or a vector comprising said polynucleotide, and (2) two or more antibodies, each modified with a DNP group, said two or more antibodies bind and/or block two or more antigens associated with a disease or condition, or two or more epitopes of an antigen associated with a disease of condition; and optionally (3) a natural killer cell or a T cell.

DNP-modified antibodies can be prepared using a coupling chemistry, especially a conjugation chemistry for antibodies. In various implementations, NHS crosslinking chemistry is used in coupling a DNP-containing molecule to an antibody. For example, N-Succinimidyl N-(2,4-dinitrophenyl)-6-aminocaproate can react with primary amines (e.g., lysine) in at pH 7-9 to form amide bonds, thereby conjugating a DNP group on an antibody. Besides NHS (N-hydroxysuccinimide ester) chemistry, a sulfhydryl-maleimide chemistry can also be used to couple a maleimide-containig DNP molecule with a cysteine-containing protein molecule. In some aspects, the DNP-modified bNAb or DNP-modified antibody includes about two, three, four, five, six, or more DNP groups per molecule of protein. In some aspects, the DNP-modified bNAb or DNP-modified antibody includes at least one DNP group per molecule of protein.

In various implementations, the combination or system disclosed herein including a genetically engineered NK cell or T cell to express a DNP-specific chimeric antigen receptor and at least one DNP-modified antibody, induces at least 10%, 20%, 30%, 40%, or 50% more cytotoxicity to an HIV-infected cell, compared to a genetically engineered NK cell or T cell alone or a non-transduced NK cell or T cell alone, or compared to the antibody alone or the DNP-modified antibody alone.

In various implementations, the combination or system disclosed herein including a genetically engineered NK cell or T cell to express a DNP-specific chimeric antigen receptor and two or more DNP-modified HIV-specific NAbs, induces at least 10%, 20%, 30%, 40%, or 50% more cytotoxicity to an HIV-infected cell, compared to a combination including a genetically engineered NK cell or T cell and only one of the DNP-modified HIV-specific NAbs or an isotype control antibody not specific for HIV.

In some embodiments, the DNP-modified antibodies in the combination or system target one or more antigens (or one or more epitopes thereof) specific for a cancer, a B-cell lineage malignancy, an inflammatory disease, a neuronal disorder, a diabetes, a cardiovascular disease, or an infectious disease. Neutralizing antibodies, or blocking antibodies, against biomarkers in each disease or condition will be apparent to those of skill in the art and may be modified with a DNP group via one or more conjugation chemistry, and to be used in connection with alternate embodiments of the invention. For example, an effective amount of DNP-modified neutralizing antibody cocktails (e.g., casirivimab/imdevimab; bamlanivimab/etesevimab; or bamlanivimab) against SARS-CoV-2 can be used in connection with an effective quantity of NK cells modified to express a DNP-specific chimeric antigen receptor for administration to a subject infected with SARS-CoV-2.

In some embodiments, an additional anti-HIV agent is included in a system or combination, or is co-administered to the subject being treated by a method disclosed herein. Such compounds include, for example, agents such as nucleoside reverse transcriptase inhibitors (NRTI), non-nucloeoside reverse transcriptase inhibitors, protease inhibitors and fusion inhibitors. Exemplary anti-HIV compounds include, for example, Amprenivir, Abacavir, Acemannan, Acyclovir, AD-439, AD-519, Adefovir dipivoxil, Alpha Interferon, Ansamycin, 097, AR 177, Beta-fluoro-ddA, BMS-232623 (CGP-73547), BMS-234475 (CGP-61755), CI-1012, Cidofovir, Curdlan sulfate, Cytomegalovirus Immune globin, Ganciclovir, Dideoxyinosine, DMP-450, Efavirenz (DMP-266), EL10, Famciclovir, FTC, GS 840, HBY097, Hypericin, Recombinant Human Interferon Beta, Interferon alfa-n3, Indinavir, ISIS-2922, KNI-272, Lamivudine (3TC), Lobucavir, Nelfinavir, Nevirapine, Novapren, Peptide T Octapeptide Sequence, Trisodium Phosphonoformate, PNU-140690, Probucol, RBC-CD4, Ritonavir, Saquinavir, Valaciclovir, Virazole Ribavirin, VX-478, Zalcitabine, Zidovudine (AZT), Tenofovir diisoproxil fumarate salt, Combivir, Abacavir succinate, T-20), AS-101, Bropirimine, CL246, EL10, FP-21399, Gamma Interferon, Granulocyte Macrophage Colony Stimulating Factor (GM-CSF), HIV Core Particle Immunostimulant, Interleukin-2 (IL-2), Immune Globulin Intravenous, IMREG-1, IMREG-2, Imuthiol Diethyl Dithio Carbamate, Alpha-2 Interferon, Methionine-Enkephalin, MTP-PE (Muramyl-Tripeptide), Granulocyte Colony Stimulating Factor (GCSF), Remune, rCD4 (Recombinant Soluble Human CD4-IgG), rCD4-IgG Hybrids, Recombinant Soluble Human CD4, Interferon Alfa 2a, SK&F1-6528, Soluble T4, Thymopentin, Tumor Necrosis Factor (TNF), AK602, Alovudine, Amdoxovir, AMD070, Atazanavir (Reyataz), AVX754 (apricitabine), Bevirimat, BI-201, BMS-378806, BMS-488043, BMS-707035, C31G, Carbopol 974P, Calanolide A, Carrageenan, Cellulose sulfate, Cyanovirin-N, Darunavir, Delavirdine, Didanosine (Videx), Efavirenz, Elvucitabine, Emtricitabine, Fosamprenavir (Lexiva), Fozivudine tidoxil, GS 9137, GSK-873,140 (aplaviroc), GSK-364735, GW640385 (brecanavir), HG0004, HGTV43, INCB9471, KP-1461, Lopinavir, Mifepristone (VGX410), MK-0518, PPL-100, PRO 140, PRO 542, PRO 2000, Racivir, SCH-D (vicriviroc), SPO1A, SPL7013, TAK-652, Tipranavir (Aptivus), TNX-355, TMC125 (etravirine), UC-781, UK-427,857 (Maraviroc), Valproic acid, VRX496, Zalcitabine, Valganciclovir, Clindamycin with Primaquine, Fluconazole Pastille, Nystatin Pastille, Eflornithine, Pentamidine, Isethionate, Trimethoprim, Trimethoprim/sulfa, Piritrexim, Pentamidine isethionate, Spiramycin, Intraconazole-R51211, Trimetrexate, Daunorubicin, Recombinant Human Erythropoietin, Recombinant Human Growth Hormone, Megestrol Acetate, Testosterone, Aldesleukin (Proleukin), Amphotericin B, Azithromycin (Zithromax), Calcium hydroxyapatite, Doxorubicin, Dronabinol, Entecavir, Epoetin alfa, Etoposide, Fluconazole, Isoniazid, Itraconazole (Sporanox), Megestrol, Paclitaxel (Taxol), Peginterferon alfa-2, Poly-L-lactic acid (Sculptra), Rifabutin (Mycobutin), Rifampin, Somatropin and Sulfamethoxazole/Trimethoprim. Preferred anti-HIV compounds for use in the present invention include, for example, 3TC (Lamivudine), AZT (Zidovudine), (-)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof.

### Pharmaceutical Compositions

Various embodiments provide pharmaceutical compositions comprising a pharmaceutically acceptable excipient and a therapeutically effective quantity of genetically engineered NK cells or T cells expressing a DNP-specific chimeric antigen receptor, or a therapeutically effective amount of a polynucleotide encoding a DNP-specific chimeric antigen receptor.

Some embodiments provide the pharmaceutical compositions further comprises at least one DNP-modified antibody.

In some embodiments, a pharmaceutical composition comprising a pharmaceutically acceptable excipient and a therapeutically effective quantity of genetically engineered NK cells or T cells expressing a DNP-specific chimeric antigen receptor, and a pharmaceutical composition comprising a pharmaceutically acceptable excipient and a therapeutically effective amount of at least one DNP-modified HIV-specific NAb, are provided in a system.

In other embodiments, a pharmaceutical composition comprising a pharmaceutically acceptable excipient and a therapeutically effective amount of a polynucleotide encoding a DNP-specific chimeric antigen receptor, a pharmaceutical composition comprising a pharmaceutically acceptable excipient and a therapeutically effective quantity of NK cells or T cells, and a pharmaceutical composition comprising a pharmaceutically acceptable excipient and a therapeutically effective amount of at least one DNP-modified HIV-specific NAb, are provided in a system.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

In various embodiments, the pharmaceutical compositions according to the invention may be formulated for delivery via any route of administration. “Route of administration” may refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly.

### Methods of Using

Various embodiments provide a method for inducing apoptosis of HIV-infected cells, said HIV-infected cells expressing HIV-1 envelop glycoprotein gp160 or gp120 and gp41, the method comprising: contacting a quantity of genetically engineered immune cells expressing a DNP-specific chimeric antigen receptor with the HIV-infected cells in the presence of a DNP-modified antibody, said DNP-modified antibody has a binding affinity specific for gp160, gp120, and/or gp41.

In some aspects, the method for inducing apoptosis of HIV-infected cells is an ex vivo method. In some aspects of the ex vivo method, the HIV-infected cells are obtained from a subject infected with HIV. In further aspects, the ex vivo method for inducing apoptosis of HIV-infected cells includes contacting a quantity of genetically engineered immune cells expressing a DNP-specific chimeric antigen receptor with the HIV-infected cells in a number ratio of at least 5:1, or more preferably between 5:1 and 100:1, or about 10:1, 15:1, 20:1, or 25:1, or 30:1, 40:1, or 50:1, in the presence of a DNP-modified antibody at a concentration of at least 1 nM, or more preferably at least 2 nM, or in a range between 2 nM and 50 nM. In other aspects, the method of inducing apoptosis of HIV-infected cells is an in vivo method. In various aspects, the method of inducing apoptosis of HIV-infected cells leads to an increased expression of IFN-γ, compared to the level in the absence of a DNP-modified antibody.

In some embodiments, a method for inducing apoptosis of HIV-infected cells, said HIV-infected cells expressing HIV-1 envelop glycoprotein gp160 or gp120 and gp41, the method comprises: contacting a quantity of NK cells expressing a DNP-specific chimeric antigen receptor with the HIV-infected cells in the presence of at least one DNP-modified, HIV-specific bNAb. In some aspects, the HIV-infected cells comprise HIV-infected human CD4⁺ T cells.

In further embodiments, a method for inducing apoptosis of HIV-infected cells, said HIV-infected cells expressing HIV-1 envelop glycoprotein gp160 or gp120 and gp41, the method comprises: contacting a quantity of NK cells expressing a DNP-specific chimeric antigen receptor with the HIV-infected cells in the presence of one or a cocktail of DNP-modified, HIV-specific bNAbs, wherein the DNP-modified, HIV-specific bNAbs comprise any one, or two or more of PG9, PGT145, PG16, 2G12, PGT128, 10-1074, and 3BNC117. In a further aspect, this method is for inducing apoptosis of HIV-infected cells expressing subtype B gp160 (or its cleavage product gp120, gp41), and the DNP-modified, HIV-specific bNAbs do not include any one or more of VRC01, b12, 10E8, 4E10 and 2F5.

Yet a method for inducing apoptosis of HIV-infected cells, said HIV-infected cells expressing subtype C gp160 (or its cleavage product gp120, gp41), comprises: contacting a quantity of NK cells expressing a DNP-specific chimeric antigen receptor with the HIV-infected cells in the presence of one or a cocktail of DNP-modified, HIV-specific bNAbs, wherein the DNP-modified, HIV-specific bNAbs comprise any one, or two or more of PG9, 10-1074, 3BNC117, and VRC01. In a further aspect of this method, the DNP-modified, HIV-specific bNAbs do not include either one or both of 2G12 and 10E8.

In another embodiment, a method for inducing apoptosis of HIV-infected cells, said HIV-infected cells expressing subtype B or subtype C gp160 (or its cleavage product gp120, gp41), comprises: detecting subtype B gp160 in the HIV-infected cells, and contacting a quantity of NK cells expressing a DNP-specific chimeric antigen receptor with the HIV-infected cells detected with subtype B gp160 in the presence of one or a cocktail of DNP-modified, HIV-specific bNAbs comprising any one, or two or more of PG9, PGT145, PG16, 2G12, PGT128, 10-1074, and 3BNC117; or detecting subtype C gp160 in the HIV-infected cells, and contacting a quantity of NK cells expressing a DNP-specific chimeric antigen receptor with the HIV-infected cells detected with subtype C gp160 in the presence of one or a cocktail of DNP-modified, HIV-specific bNAbs comprising any one, or two or more of PG9, 10-1074, 3BNC117, and VRC01.

Other embodiments provide a method for inducing apoptosis of malignant B cells, said malignant B cells overexpressing CD19 compared to a normal, non-malignant B cells, and the method comprises: contacting a quantity of NK cells expressing a DNP-specific chimeric antigen receptor with the malignant B cells in the presence of a DNP-modified, anti-CD19 antibody. In some aspects, the malignant B cells are associated with or also infected with HIV, and the method comprises contacting a quantity of NK cells expressing a DNP-specific chimeric antigen receptor with the malignant B cells in the presence of a DNP-modified, HIV-specific bNAb; optionally further in the presence of a DNP-modified, anti-CD19 antibody.

Additional embodiments provide a method for treating a subject suffering from acquired immunodeficiency syndrome (AIDS) or infected with HIV, comprising: administering to the subject a therapeutically effective quantity of genetically engineered immune cells expressing a DNP-specific chimeric antigen receptor and a therapeutically effective amount of at least one DNP-modified, HIV-specific antibody.

In some aspects of the method for treating the subject suffering from AIDS or infected with HIV, the method includes administering to the subject a therapeutically effective quantity of genetically engineered NK cells expressing a DNP-specific chimeric antigen receptor and a therapeutically effective amount of two or more DNP-modified, HIV-specific bNAbs.

In other aspects of the method for treating the subject suffering from AIDS or infected with HIV, the method includes administering to the subject a therapeutically effective quantity of genetically engineered T cells expressing a DNP-specific chimeric antigen receptor and a therapeutically effective amount of two or more DNP-modified, HIV-specific bNAbs.

In some aspects, the methods further include selecting a subject infected with HIV. In various aspects, the subject is a human.

Other embodiments provide a method for treating a subject suffering from AIDS or infected with HIV, comprising: transfecting a quantity of NK cells or T cells with a polynucleotide encoding a DNP-specific chimeric antigen receptor, wherein the NK cells or the T cells are not infected with the HIV, thereby obtaining transfected NK cells or transfected T cells, followed by transplanting the transfected NK cells or transfected T cells to the subject, and administering a therapeutically effective amount of at least one DNP-modified, HIV-specific antibody, so as to induce apoptosis of HIV-infected cells in the subject.

Further embodiments provide a method of treating a subject infected with HIV or suffering from AIDS, comprising:
detecting expression of subtype B gp160 in HIV-infected cells of the subject, and administering one or a cocktail of DNP-modified, HIV-specific bNAbs to the subject detected with the expression of subtype B gp160 in the HIV-infected cells, wherein the one or cocktail of DNP-modified, HIV-specific bNAbs comprise any one, or two or more of PG9, PGT145, PG16, 2G12, PGT128, 10-1074, and 3BNC117, and wherein the subject has been or is transplanted with a quantity of NK cells or T cells expressing a DNP-specific chimeric antigen receptor; or
detecting expression of subtype C gp160 in HIV-infected cells of the subject, and administering one or a cocktail of DNP-modified, HIV-specific bNAbs to the subject detected with the expression of subtype B gp160 in the HIV-infected cells, wherein the one or cocktail of DNP-modified, HIV-specific bNAbs comprise any one, or two or more of PG9, 10-1074, 3BNC117, and VRC01, and wherein the subject has been or is transplanted with a quantity of NK cells or T cells expressing a DNP-specific chimeric antigen receptor.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Here, we report the development of a chemically-directed universal CAR-NK cell capable of recognizing various epitopes of gp160 from different HIV-1 subtypes. Instead of targeting gp160 directly, this CAR recognizes 2,4-dinitrophenyl (DNP), a small-molecule ligand, and its antigen specificity can subsequently be redirected to target different epitopes of HIV-1 gp160 variants using DNP-conjugated antibodies as adaptor molecules. As a proof of concept, we show that in the presence of adaptor molecules, anti-DNP CAR-NK cells can specifically target and kill mimic HIV-infected cell lines expressing gp160 of subtypes B and C. We additionally find that adaptor molecules targeting membrane-distal epitopes, which have higher accessibility, are more likely to activate anti-DNP CAR-NK cells against gp160+ target cells compared with those targeting membrane-proximal epitopes. This modular universal CAR-NK cell platform, combined with numerous readily-available anti-gp160 antibodies, can potentially be used to overcome HIV-1 diversity and enhance the eradication of infected cells, which is essential to ultimately finding a universal cure for HIV.

This study is the first to engineer NK cells with a universal CAR and to apply it for targeting HIV-1 epitopes. It is important to note that allogeneic T cells from healthy donors carry a high risk of inducing graft-versus-host-disease (GVHD) due to the expression of highly diverse T cell receptors, while allogeneic NK cells have little or no such risk. Therefore, the universal CAR-NK cell can potentially be developed as an off-the-shelf cellular therapeutic for all patients.

### Genetic Engineering of NK cells to Express an Anti-DNP CAR

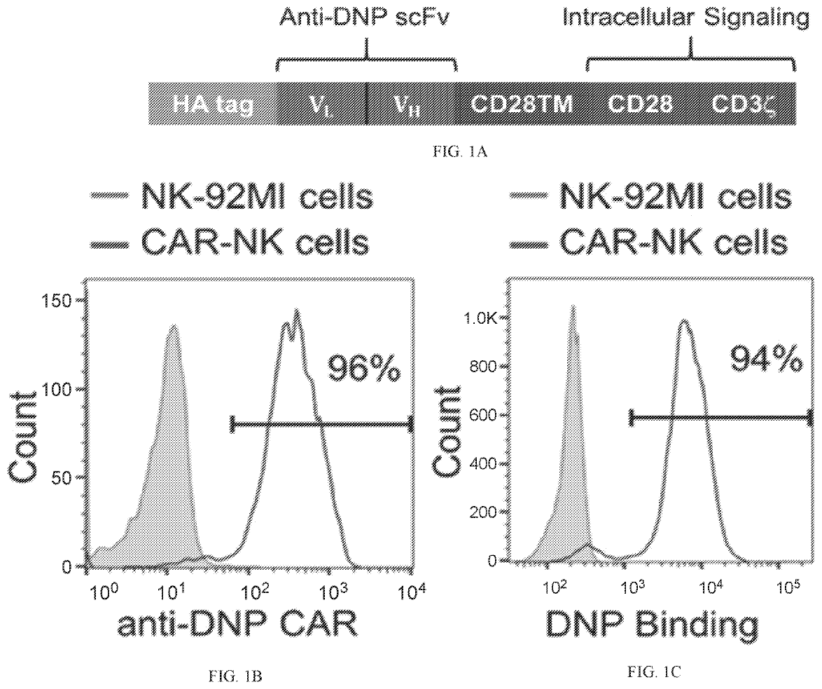

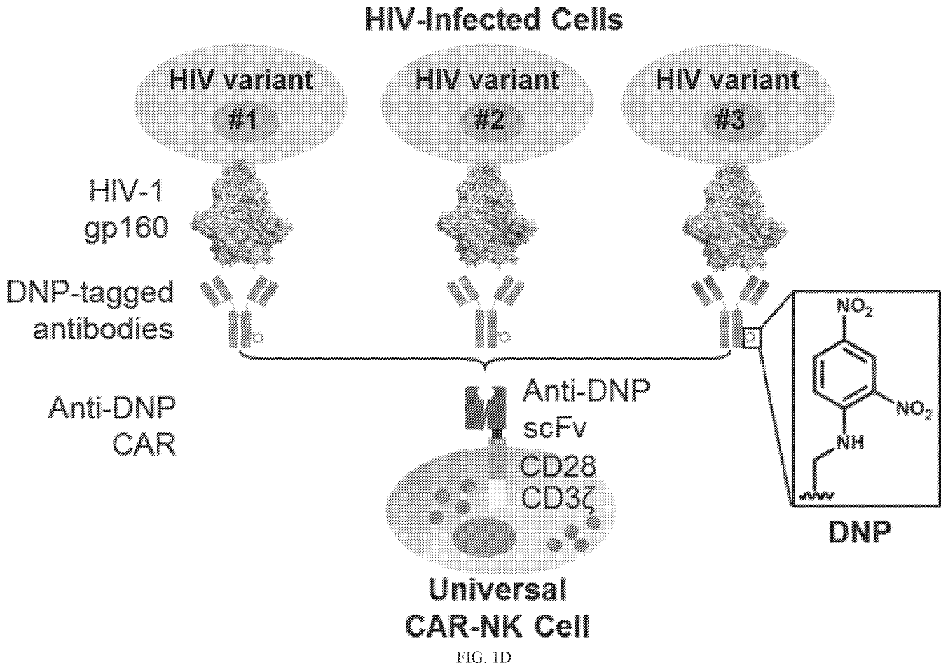

We started by designing a DNP-mediated universal CAR. We chose DNP as the target of the CAR for three reasons. First, the sequences of many anti-DNP antibodies are available for CAR design. Second, DNP-conjugated anti-gp160 antibodies can be generated by chemical conjugation. Third, DNP is biocompatible. As depicted in FIG. 1A, an exemplary anti-DNP CAR consists of an N-terminal HA-tag for the detection of CAR expression, an extracellular anti-DNP scFv, a CD28 transmembrane domain (CD28TM), and intracellular CD28/CD3ζ signaling domains. The anti-DNP scFv was designed based on a high-affinity mouse anti-DNP antibody IgG2a-2, in the format of VL-(GGGGS)₃ (SEQ ID NO:1)-VH. The CD28/CD3ζ signaling domains have been broadly used in the design of second-generation CARs. The gene of the anti-DNP CAR was assembled by overlap extension PCR and inserted to the pFUW lentiviral vector. This construct was then used to generate lentiviral particles in HEK293T cells. We have tested at least six anti-DNP scFvs in CAR design, three of which have worked:

(1) IgG2a-2 scFv, high affinity:
DNA sequence of V_{L}:
(SEQ ID NO: 9)
5′-gatatccagatgacacagactacatcctccctgtctgcctctctggga
gacagagtcaccatcagttgcagggcaagtcaggatattagcaattattta
aactggtatcagcagaaaccagatggaactgttaaactcctgatctactac
acatcaagattacactcaggagtcccatcaaggttcagtggcagtgggtct
ggaacagattattctctcaccattagcaacctggaacaagaagatattgcc
acttacttttgccaacagggtaatacgcttccgtggacgttcggtggaggc
accaagctggaaatcaaa-3′.
DNA sequence of V_{H}:
(SEQ ID NO: 10)
5′-gatgtacgccttcaggagtcaggacctggcctcgtgaaaccttctcag
tctctgtctctcacctgctctgtcactggctactccatcaccaatagttat
tactggaactggattcggcagtttccaggaaacaaactggaatggatggtc
tacataggctacgacggtagcaataactacaacccatctctcaaaaatcga
atctccatcactcgtgacacatctaagaaccagtttttcctgaagttgaac
tctgtgactactgaggacacagctacatattactgtgcaagagctacctac
tatggtaactacagggggtttgcttactggggccaagggactctggtcact
gtctctgca-3′.
Amino acid sequence of V_{L}:
(SEQ ID NO: 11)
DIQMTQTTSSLSASLGDRVTISCRASQDISNYLNWYQQKPDGTVKLLIYYT
SRLHSGVPSRFSGSGSGTDYSLTISNLEQEDIATYFCQQGNTLPWTFGGGT
KLEIK.
Amino acid sequence of V_{H}:
(SEQ ID NO: 12)
DVRLQESGPGLVKPSQSLSLTCSVTGYSITNSYYWNWIRQFPGNKLEWMVY
IGYDGSNNYNPSLKNRISITRDTSKNQFFLKLNSVTTEDTATYYCARATYY
GNYRGFAYWGQGTLVTVSA.
(2) IgG2a-6 scFv, medium affinity:
DNA sequence of V_{L}:
(SEQ ID NO: 13)
5′-gatgttgtgatgacccaaactccactctccctgcctgtcagtcttgga
gatcaagcctccatctcttgcagatctagtcagagtcttgtacacagtaat
ggaaacacctatttacattggaacctgcagaagccaggccagtctccaaag
ctcctgatctacaaagtttccaaccgattttctggggtcccagacaggttc
agtggcagtggatcagggacagatttcacactcaagatcagcagagtggag
gctgaggatctgggagtttatttctgctctcaaagtacacatgttccgtgg
acgttcggtggaggcaccaagctggaaatcaaa-3′.
DNA sequence of V_{H}:
(SEQ ID NO: 14)
5′-gaggtgcagcttcaggagtcaggacctagcctcgtgaaaccttctcag
actctgtccctcacctgttctgtcactggcgactccatcaccagtggttac
tggaactggctccggaaattcccagggattaaatttgagtacatggggtac
ataagttacagtggtagcacttactacaatccatctctcaaaagtcgaatc
tccatcactcgagacacatccaagaaccagtactacctgcagttgaactct
gtgactaatgaggacacagccacatattactgtgcaagatactatggtaac
ggctatgctttggactactggggtcaaggaacctcagtcaccgtctcctc
a-3′.
Amino acid sequence of V_{L}:
(SEQ ID NO: 15)
DVVMTQTPLSLPVSLGDQASISCRSSQSLVHSNGNTYLHWNLQKPGQSPKL
LIYKVSNRFSGVPDRFSGSGSGTDFTLKISRVEAEDLGVYFCSQSTHVPWT
FGGGTKLEIK.
Amino acid sequence of V_{H}:
(SEQ ID NO: 16)
EVQLQESGPSLVKPSQTLSLTCSVTGDSITSGYWNWLRKFPGIKFEYMGYI
SYSGSTYYNPSLKSRISITRDTSKNQYYLQLNSVTNEDTATYYCARYYGNG
YALDYWGQGTSVTVSS.
(3) AN02 scFv, low affinity:
DNA sequence of V_{L}:
(SEQ ID NO: 17)
5′-cagattgtgctgacccagagcccggcgattatgagcgcgagcccgggc
gaaaaagtgaccatgacctgcagcgcgagcagcagcgtgtattatatgtat
tggtatcagcagaaaccgggcagcagcccgcgcctgctgatttatgatacc
agcaacctggcgagcggcgtgccggtgcgctttagcggcagcggcagcggc
accagctatagcctgaccattagccgcatggaagcggaagatgcggcgacc
tattattgccagcagtggagcagctatccgccgattacctttggcgtgggc
accaaactggaactgaaa-3′.
DNA sequence of V_{H}:
(SEQ ID NO: 18)
5′-gatgtgcagctgcaggaaagcggcccgggcctggtgaaaccgagccag
agccagagcctgacctgcaccgtgaccggctatagcattaccagcgattat
gcgtggaactggattcgccagtttccgggcaacaaactggaatggatgggc
tatatgagctatagcggcagcacccgctataacccgagcctgcgcagccgc
attagcattacccgcgataccagcaaaaaccagttttttctgcagctgaaa
agcgtgaccaccgaagataccgcgacctatttttgcgcgcgcggctggccg
ctggcgtattggggccagggcacccaggtgagcgtgagc-3′.
Amino acid sequence of V_{L}:
(SEQ ID NO: 19)
QIVLTQSPAIMSASPGEKVTMTCSASSSVYYMYWYQQKPGSSPRLLIYDTS
NLASGVPVRFSGSGSGTSYSLTISRMEAEDAATYYCQQWSSYPPITFGVGT
KLELK.
Amino acid sequence of V_{H}:
(SEQ ID NO: 20)
DVQLQESGPGLVKPSQSQSLTCTVTGYSITSDYAWNWIRQFPGNKLEWMGY
MSYSGSTRYNPSLRSRISITRDTSKNQFFLQLKSVTTEDTATYFCARGWPL
AYWGQGTQVSVS.

Next, we engineered NK-92 cells, a highly cytotoxic human NK cell line, to express anti-DNP CARs. Compared with allogeneic T cells, NK cells have the advantage of having little or no risk of inducing graft-versus-host diseases (GVHD). There is thus an increasing interest in engineering CAR-NK cells, including both primary cells and cell lines, as potential off-the-shelf cellular therapeutics. We chose NK-92 as the host cell because it can be continuously expanded to provide an unlimited number of homogenous cells, and CAR-engineered NK-92 cells have been safely utilized in clinical trials against cancer. Since the growth of NK-92 cells is dependent on interleukin 2 (IL-2), we used an IL-2-secreting NK-92-derived cell line (NK-92MI) as the host cell for CAR engineering. NK-92MI cells were transduced with lentiviral particles generated above to express anti-DNP CARs. The CAR expression was confirmed by staining with an anti-HA-tag antibody and a phycoerythrin (PE)-conjugated secondary antibody, followed by flow cytometric analysis. The initial lentiviral transduction led to approximately 3% CAR-expressing cells, which were subsequently enriched to 96% purity by magnetic cell sorting (FIG. 1B). To verify whether the extracellular anti-DNP scFv was expressed in the functional conformation, CAR-NK cells were stained with a homemade DNP-conjugated fluorescent protein PE. Flow cytometric analysis showed that approximately 94% of cells were stained positively, confirming that they were able to recognize DNP (FIG. 1C).

### Redirection of Anti-DNP CAR-NK Cells to Target Subtype B gp160⁺ Cells Using a DNP-conjugated Antibody

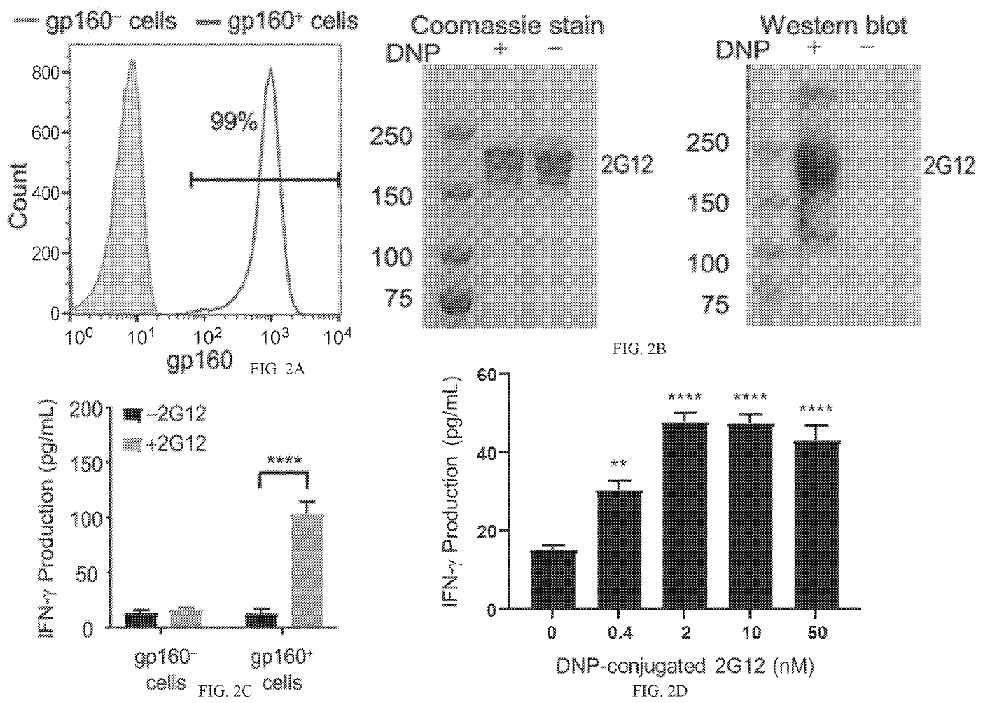

Because HIV-infected cells express the viral envelope glycoprotein gp160 at the cell surface, we developed a gp160-expressing cell line as a mimic of HIV-infected cells to be targeted by CAR-NK cells. The cell line was generated by transfecting HEK293 cells with the vector pConBgp160-opt, which encodes a full-length subtype B consensus gp160. The expression of gp160 was confirmed by cell surface staining using a human anti-gp120 antibody VRC01 and a PE-conjugated anti-human IgG polyclonal antibody. After transfection using Lipofectamine 2000 (a lipid-based transfection reagent), approximately 15-20% of cells expressed gp160, which were selected by G418 (Geneticin) and then further enriched to a purity of 99% by fluorescence-activated cell sorting (FACS) (FIG. 2A).

Next, we generated a DNP-conjugated anti-gp160 antibody as the adaptor molecule. We chose 2G12, a bNAb that recognizes the high-mannose glycan epitope proximal to the variable region V3 of gp160. DNP conjugation was conducted using N-(2,4-dinitrophenyl)-6-aminocaproic acid N-succinimidyl ester (“DNP-NHS ester”; N-Succinimidyl N-(2,4-dinitrophenyl)-6-aminocaproate). The attachment of DNP to the antibody was verified by SDS-PAGE, followed by western blot analysis (FIG. 2B). The conjugate contained approximately two DNP moieties per antibody, as determined by measuring the absorbance at 280 nm (for the antibody) and 360 nm (for DNP).

To test the feasibility of using DNP-conjugated 2G12 to redirect anti-DNP CAR-NK cells against gp160⁺ cells, we used an in vitro co-culture assay. Specifically, anti-DNP CAR-NK cells were co-cultured overnight with subtype B gp160⁺ HEK293 cells at an effector-to-target (E:T) ratio of 1:1, with or without the DNP-conjugated antibody 2G12 (10 nM). In the negative control, CAR-NK cells were co-cultured with plain, gp160-negative HEK293 cells. The activation of CAR-NK cells was assessed by measuring the secretion of interferon-γ (IFN-γ) using an enzyme-linked immunosorbent assay (ELISA). As expected, gp160-negative cells could not activate anti-DNP CAR-NK cells, regardless of whether DNP-conjugated 2G12 was present or not. Subtype B gp160⁺ cells alone could not activate CAR-NK cells either, but they became stimulatory in the presence of DNP-conjugated 2G12, inducing CAR-NK cells to secrete a significant amount of IFN-γ (FIG. 2C). Together, these results indicate that anti-DNP CAR-NK cells can be redirected to target gp160⁺ cells.

To determine the optimal concentration of adaptor molecules, we cultured CAR-NK cells and gp160⁺ target cells with DNP-conjugated 2G12 at different concentrations (0, 0.4, 2, 10, and 50 nM). We found that IFN-γ production reached a plateau when the 2G12 adaptor molecule was used at 2 nM or above (FIG. 1D). Based on this result, we used 2G12 and other bNAb-derived adaptor molecules at 2 nM in all subsequent experiments.

### Anti-gp160 Antibodies Targeting Membrane-distal Epitopes are More Likely to Activate CAR-NK Cells Compared with Those Targeting Membrane-proximal Epitopes

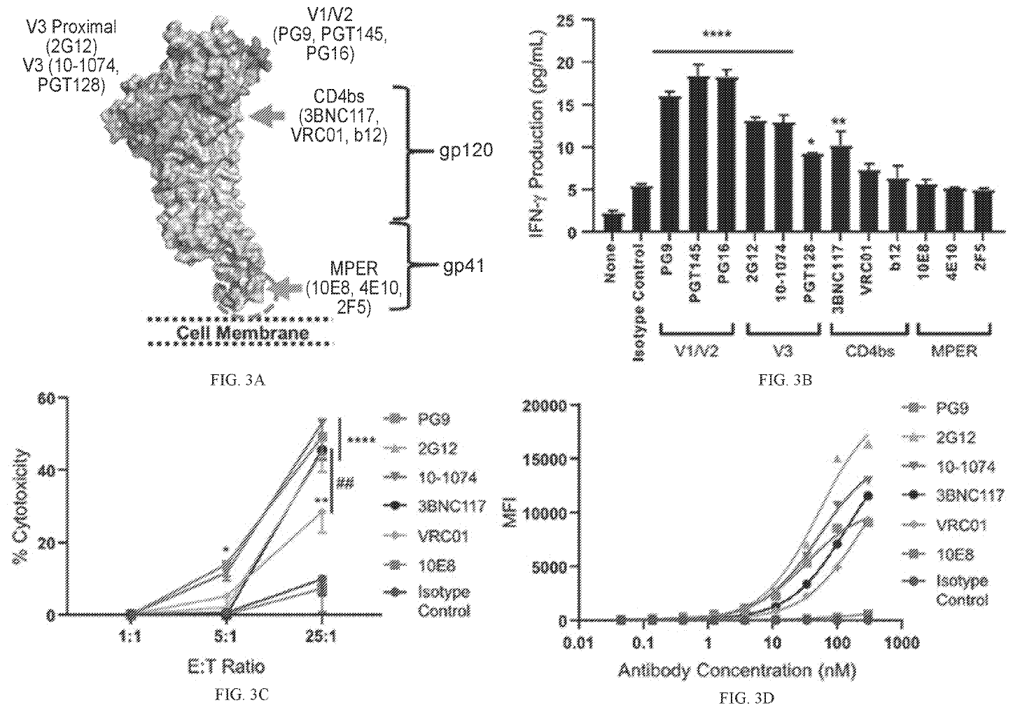

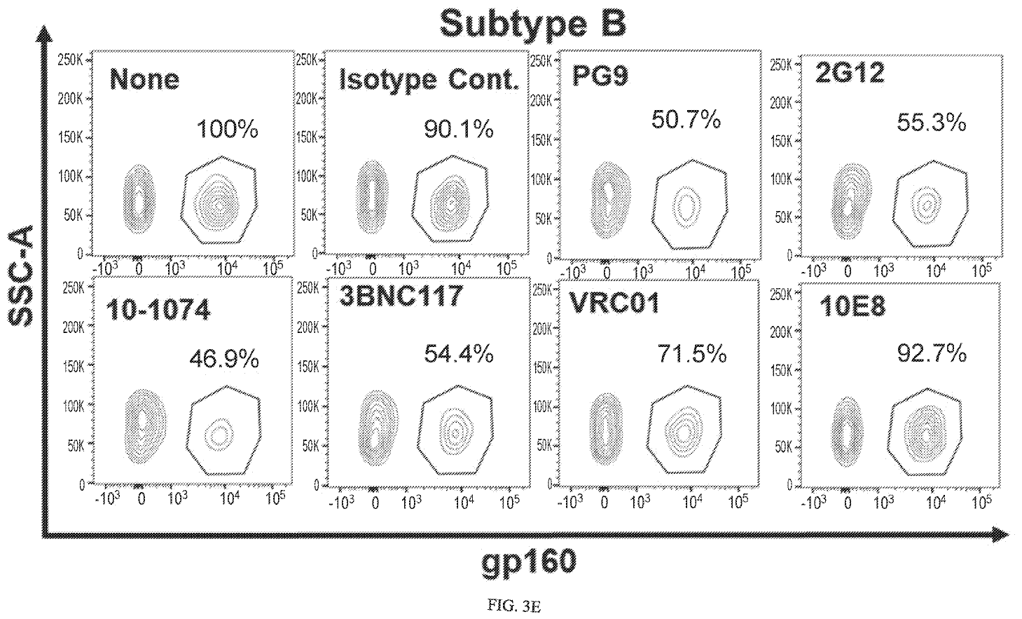

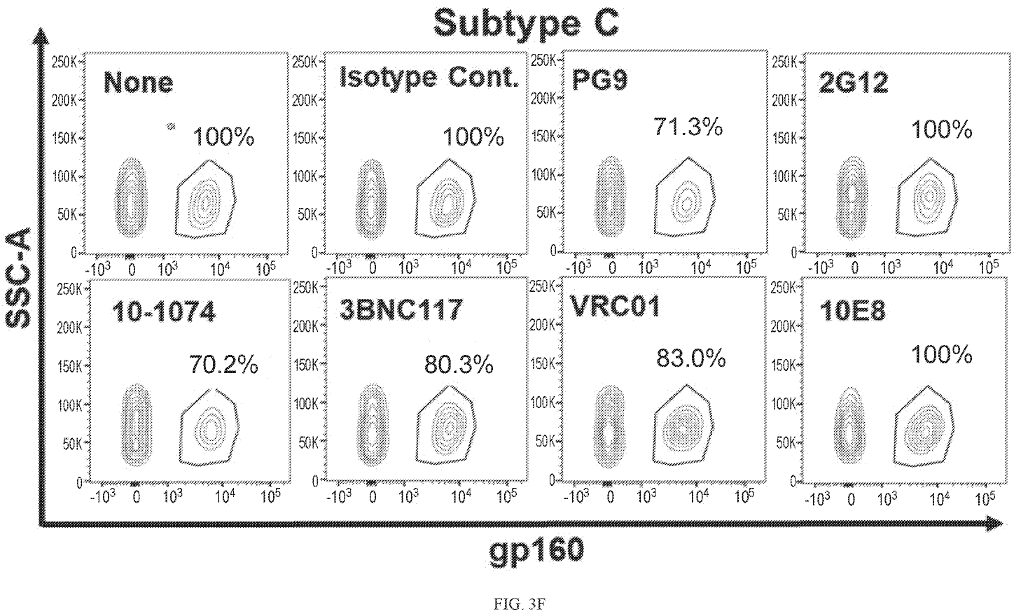

Numerous bNAbs recognizing different epitopes of HIV-1 gp160 have previously been identified. We asked whether the epitope location on gp160 could affect the ability of a bNAb to redirect anti-DNP CAR-NK cells against gp160⁺ cells. To address this question, we selected 12 well-characterized bNAbs recognizing four different regions of gp160, including: 1) PG9, PGT145, and PG16, which recognize glycan epitopes in the V1/V2 region of gp120; 2) 2G12, PGT128, and 10-1074, which recognize glycan epitopes in or proximal to the V3 region of gp120; 3) VRC01, b12, and 3BNC117, which recognize the CD4-binding site (CD4bs) on gp120; and 4) 10E8, 4E10, and 2F5, which recognize the gp41 membrane-proximal external region (MPER) (FIG. 3A). We conjugated these antibodies with DNP, and then compared their ability to redirect anti-DNP CAR-NK cells against subtype B gp160⁺ target cells using in vitro co-culture assays.

We first assessed the levels of CAR-NK cell activation using IFN-γ production assays. CAR-NK cells and gp160⁺ cells at a 1:1 ratio were incubated with DNP-conjugated bNAbs (2 nM). As negative controls, cells were either incubated with DNP-conjugated human IgG (isotype control) or without any antibodies. After four hours, the concentration of IFN-γ in the culture supernatant was measured by ELISA. We found that all three V1/V2-targeting antibodies—PG9, PGT145, and PG16—were able to strongly activate CAR-NK cells against gp160⁺ target cells, leading to a three- to four-fold increase in IFN-γ production as compared with cells incubated with the isotype control (FIG. 3B). The three V3-targeting antibodies—2G12, PGT128, and 10-1074—also significantly activated CAR-NK cells, leading to a two- to threefold increase in IFN-γ production. Among the three CD4bs antibodies, 3BNC117 activated CAR-NK cells to produce IFN-γ about two-fold more than the isotype control, but VRC01 and b12 did not significantly activate CAR-NK cells. Strikingly, none of the three MPER-binding antibodies—10E8, 4E10, and 2F5—could activate CAR-NK cells against subtype B gp160⁺ target cells.

Next, we assessed CAR-NK cell-mediated killing of gp160⁺ cells using a flow cytometry-based cytotoxicity assay. For ease of detection, subtype B gp160⁺ cells were pre-labeled with fluorescein using carboxyfluorescein succinimidyl ester. These cells were mixed with non-labeled gp160-negative cells at a 1:1 ratio, and then co-cultured with anti-DNP CAR-NK cells at varying E:T ratios (1:1, 5:1, and 25:1), with or without DNP-conjugated bNAbs (2 nM). Eight hours later, cells were stained with a viability dye and subjected to flow cytometric analysis. The levels of cytotoxicity were determined based on the percentage of gp160⁺ cells killed by CAR-NK cells. We selected six bNAbs to be tested in the cytotoxicity assay, including 1) PG9, 2G12, 10-1074, and 3BNC117, which were effective in the IFN-γ assay, and 2) VRC01 and 10E8, which were ineffective in the IFN-γ assay. We found that the first four bNAbs were also more potent in the cytotoxicity assay, activating CAR-NK cells to kill approximately 50% of gp160⁺ cells after an eight-hour incubation at the 25:1 E:T ratio (FIG. 3C). VRC01 was less potent, inducing CAR-NK cells to kill 28.5% of gp160⁺ cells. The MPER-targeting bNAb, 10E8, however, did not induce any significant killing as compared with the isotype control, which was consistent with its lack of activity in the IFN-γ assay. At lower E:T ratios, the cytotoxicity of CAR-NK cells was significantly reduced. Indeed, after an eight-hour incubation, only PG9 and 10-1074 were able to activate anti-DNP CAR-NK cells to kill approximately 10-15% of gp160⁺ cells at the 5:1 E:T ratio, and no cytolysis was detectable at the 1:1 E:T ratio. Importantly, CAR-NK cells did not kill gp160-negative HEK293 cells under any conditions, indicating that the targeting was gp160-specific (FIG. 3E, 3F).

The above results indicate that the V1/V2, V3, and CD4bs-targeting antibodies have a greater likelihood of activating anti-DNP CAR-NK cells against gp160⁺ target cells than do MPER-targeting antibodies. A possible reason is that MPER-targeting bNAbs have poor epitope accessibility due to local steric hindrance and thus cannot efficiently bind to gp160 on the target cell surface. To compare the binding potency of bNAbs, we conducted a cell surface staining assay. Specifically, subtype B gp160⁺ cells were stained with bNAbs at a three-fold serial dilution (from 300 to 0.045 nM), followed with a PE-conjugated secondary antibody against human IgG. Cells were then subjected to flow cytometric analysis, and the average fluorescence intensity per cell was plotted against the concentration of bNAb. As shown in FIG. 3D, the V1/V2 and V3-targeting antibodies PG9, 10-1074, and 2G12 strongly bound to subtype B gp160. Interestingly, the saturated staining with PG9 was weaker than that of 2G12 and 10-1074. This is probably because gp160 proteins are expressed as trimers on the cell surface, and the three epitopes of PG9 within the same trimer are located close to each other, preventing the simultaneous binding of three PG9 antibodies. The two CD4bs-targeting antibodies 3BNC117 and VRC01 also positively bound to gp160⁺ cells but did not reach binding saturation within the tested concentration range (0.045-300 nM). However, the MPER-targeting antibody 10E8, even at 300 nM, could not stain gp160⁺ cells to any significant extent as compared with the isotype control, indicating that it has poor epitope accessibility. Overall, we found that the gp160-binding potency of antibody was roughly consistent with its ability to redirect anti-DNP CAR-NK cells against gp160⁺ cells (FIGS. 3B-3D). It is possible that other factors, such as the binding orientation of CARs over gp160 and the intermembrane distance between effector and target cells, can also affect the potency of CAR-NK cells, which remain to be determined in future studies.

### Redirection of Anti-DNP CAR-NK Cells to Target Subtype C gp160⁺ Cells

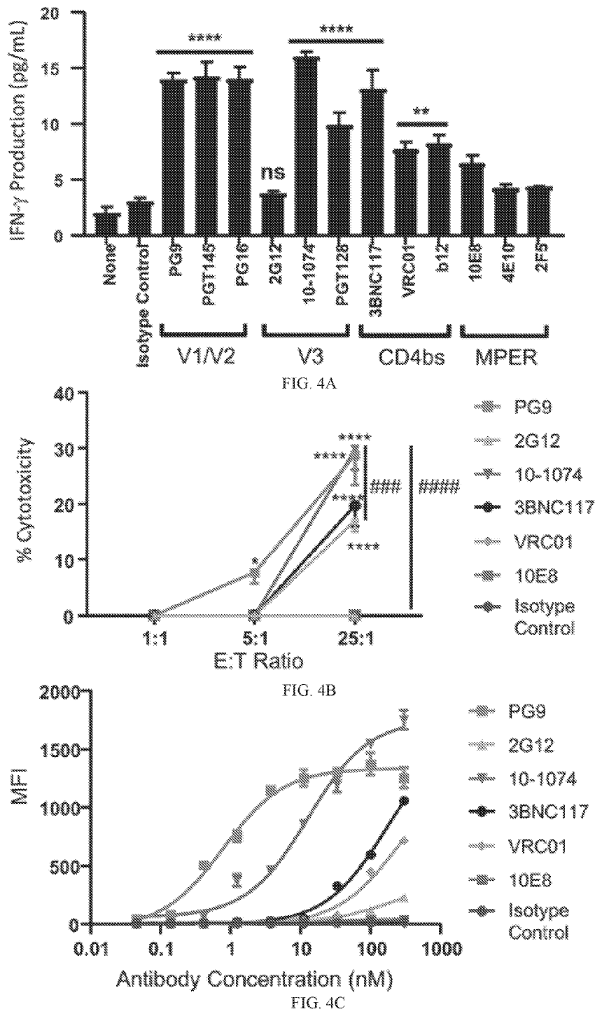

To evaluate the generality of the universal CAR-NK cell approach, we additionally used it to target HIV-1 subtype C gp160. HEK293 cells were modified to express a subtype C consensus gp160 protein using the vector pConCgp160-opt as previously reported. Subtype C gp160⁺ cells were then incubated with anti-DNP CAR-NK cells in the presence of different DNP-conjugated bNAbs (2 nM). Similar to our findings for subtype B gp160⁺ cells, we found that a majority of the nine V1/V2, V3, and CD4bs-targeting antibodies were able to strongly activate CAR-NK cells to produce IFN-γ in response to subtype C gp160⁺ cells, while none of the three MPER-targeting antibodies could significantly activate CAR-NK cells compared with the isotype control (FIG. 4A). Surprisingly, 2G12, one of the V3-targeting antibodies that were highly effective against subtype B gp160 (FIG. 3B), completely lost its ability to activate CAR-NK cells to produce IFN-γ against subtype C gp160 (FIG. 4A). The cytotoxicity assay also showed that 2G12 could not redirect CAR-NK cells to kill subtype C gp160⁺ cells, neither could the MPER-targeting antibody 10E8 and the isotype control (FIG. 4B, and FIG. 3F). For comparison, PG9 and 10-1074 redirected CAR-NK cells to kill about 30% of subtype C gp160⁺ cells after an eight-hour incubation at a 25:1 E:T ratio, and 3BNC117 and VRC01 led to lysis of about 20% of target cells. We then used these antibodies to stain subtype C gp160⁺ cells. The result showed that PG9 and 10-1074 had the highest binding potency, followed by 3BNC117 and VRC01, while 2G12, 10E8, and the isotype control exhibited little or no binding ability within the tested concentration range (0.045-300 nM) (FIG. 4C).

Therefore, as with the experiments on subtype B gp160, the relative binding potency of antibodies for subtype C gp160 was largely consistent with their ability to induce IFN-γ production and cytotoxicity of anti-DNP CAR-NK cells in response to subtype C gp160⁺ cells. Importantly, 2G12 demonstrates a dramatically different ability to activate CAR-NK cells against subtypes B and C gp160⁺ target cells, highlighting the advantage of using a universal CAR whose antigen specificity is not limited by a single antibody. We also noted that the universal CAR-NK cells were significantly more cytotoxic to subtype B than to subtype C gp160⁺ cells (FIGS. 3C and 4B). For example, with the 10-1074-derived adaptor molecule, the universal CAR-NK cells were able to kill about 53.1% of subtype B gp160⁺ cells but only 29.8% of subtype C gp160⁺ cells. Likewise, the 3BNC117-derived adaptor molecule induced CAR-NK cells to kill 45.5% of subtype B gp160⁺ cells but only 19.7% of subtype C gp160⁺ cells. The preferential targeting of subtype B over subtype C gp160 is likely because most bNAbs have been developed against subtype B HIV-1 variants. To enhance the cytotoxicity against subtype C gp160⁺ cells, it would be necessary to use other bNAbs that are more specialized for subtype C gp160.

Next, we examined whether different bNAbs could be used in combination to further enhance the efficacy of universal CAR-NK cells against a mixture of different subtypes of gp160- expressing cells. To this end, CAR-NK cells and subtypes B and C gp160⁺ cells were cocultured at a 25:1:1 ratio, in the presence of either 10-1074 (5 nM), 3BNC117 (5 nM), or both. After an 8 h incubation at 37° C., cells were stained with a viability dye (7-AAD), and the numbers of viable gp160+ cells were measured by flow cytometry. The percentages of gp160+ cells killed were found as follows: 41% with 10-1074, 31% with 3BNC117, and 60% with both of them (FIG. 8). Hence, a combination of 10-1074 and 3BNC117 was more potent than either alone in eliciting CAR-NK cell cytotoxicity against subtypes B and C gp160+ cells. This result highlights the potentiality of using universal CAR-NK cells supplemented with a cocktail of bNAbs to target diverse gp160+ cells.

### Comparison of Universal and Conventional CAR-NK Cells

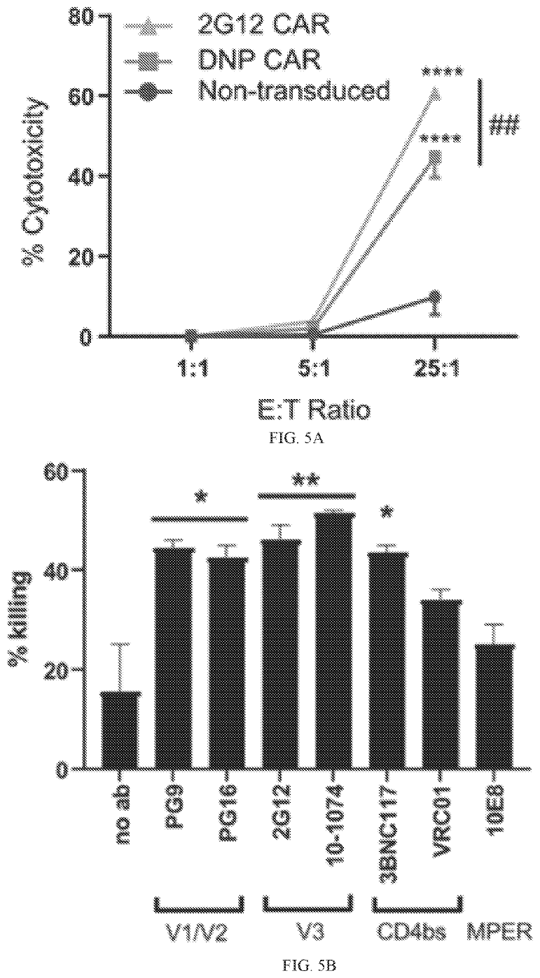

The major advantage of the universal CAR-NK cells developed above is that they can be coupled with various adaptor molecules to expand epitope coverage. However, it is unclear whether the potency of universal CAR-NK cells is comparable to that of conventional bNAb-based single-specificity CAR-NK cells. To address this question, we designed a 2G12-based anti-gp160 CAR (2G12 CAR) and generated CAR-NK cells using the same approach as described above. We then compared the cytotoxicity of universal CAR-NK cells (in combination with DNP-conjugated 2G12) and 2G12 CAR-NK cells against subtype B gp160⁺ target cells. We found that at an E:T ratio of 25:1, the universal and the conventional CAR-NK cells killed about 50% and 60% of gp160⁺ cells, respectively (FIG. 5A). At the 5:1 and 1:1 E:T ratios, neither of them was effective. These results indicate that despite having a greater epitope breadth, the universal CAR-NK cells are only slightly less potent than the conventional single-specificity CAR-NK cells.

### Killing of HIV-Infected Primary Human CD4⁺ T Cells

Next, we examined whether universal CAR-NK cells could kill HIV-infected T cells. To this end, human primary CD4+ T cells from healthy donors were stimulated with anti-CD3/CD28 beads for 2 days and were infected with HIV-1 NL4-3 (a commonly used X4-tropic subtype B HIV-1 virus) for 10 days. Infected CD4+ T cells were prestained with CellTrace Far Red, incubated with 10 nM of DNP-conjugated bNAbs for 20 min, and then cocultured with anti-DNP CAR-NK cells at a 20:1 E/T ratio for 16 h. Afterward, cells were stained with Zombie Violet (a fixable viability dye), and live HIV-infected cells were identified by intracellular staining for HIV-1 Gag. We observed an effective killing of HIV Gag-expressing CD4⁺ T cells by CAR-NK cells with various DNP-modified bNAbs as compared with the no-antibody control (FIG. 5B). In particular, we found that PG9, PG16, 2G12, 10-1074, and 3BNC117 (which target membrane-distal epitopes) were more effective than 10E8 (which targets a membrane-proximal epitope), similar to what we found with the gp160+ cell line (FIG. 3B).

### Redirection of Anti-DNP CAR-NK Cells to Target Malignant B Cells

HIV-infected patients have an approximately 60 to 200-fold higher risk of developing non-Hodgkin's lymphoma (NHL) when compared with the general population, presumably due to their compromised immune system. We speculated that the universal CAR-NK cell could be used to eradicate HIV-associated lymphoma as well. As a proof of concept, we conjugated DNP to the antibody FMC63 recognizing CD19, a biomarker overexpressed in most B-cell malignancies. We then used the conjugate to redirect anti-DNP CAR-NK cells against REH, a malignant B cell line. The flow cytometry-based cytotoxicity assay showed that at an E:T ratio of 25:1, approximately 41% of REH cells remained alive after overnight incubation, that is, 59% of cells were killed by CAR-NK cells (FIG. 7). Our universal DNP-directed CAR-NK cell platform hence provides a potential solution to eradicate both infected and malignant cells.

### The Universal CAR-NK Cell-mediated Killing of gp160⁺ Cells is not Due to ADCC

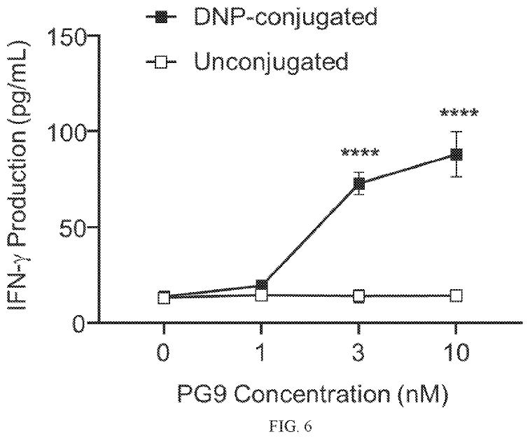

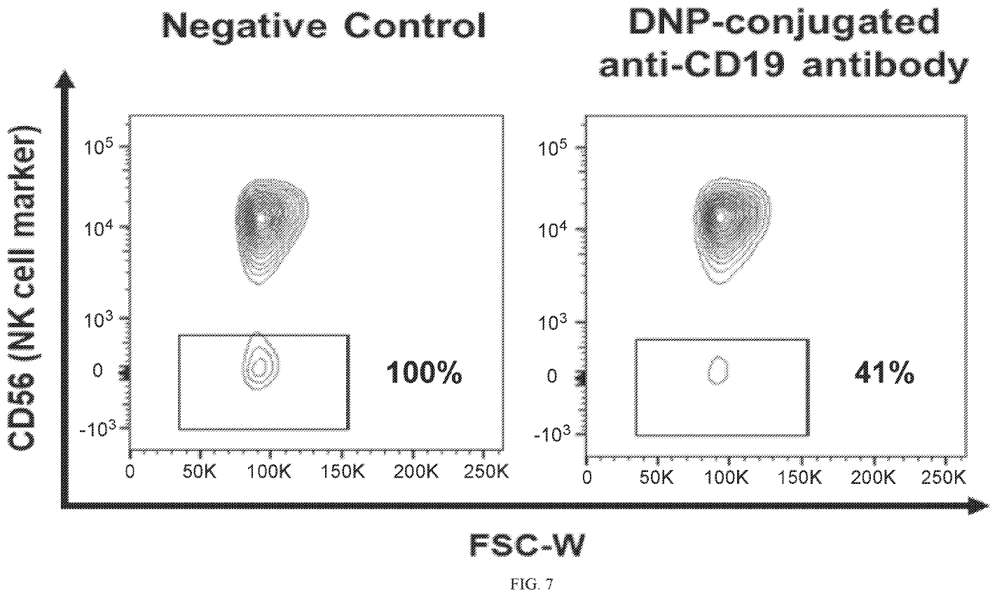

It should be noted that primary NK cells express CD16 (FcγRIII) to recognize antibodies and kill antibody-coated cells using the mechanism of antibody-dependent cellular cytotoxicity (ADCC). Since NK-92 cells do not express CD16, the CAR-NK cell-mediated killing of gp160⁺ cells observed above is not due to ADCC. This notion is consistent with our finding that DNP-conjugated PG9 can effectively activate anti-DNP CAR-NK to target subtype B gp160⁺ cells, but the unconjugated PG9 cannot (FIG. 6).

### Engineering Anti-DNP CAR-T Cells to Target gp160⁺ Cells.

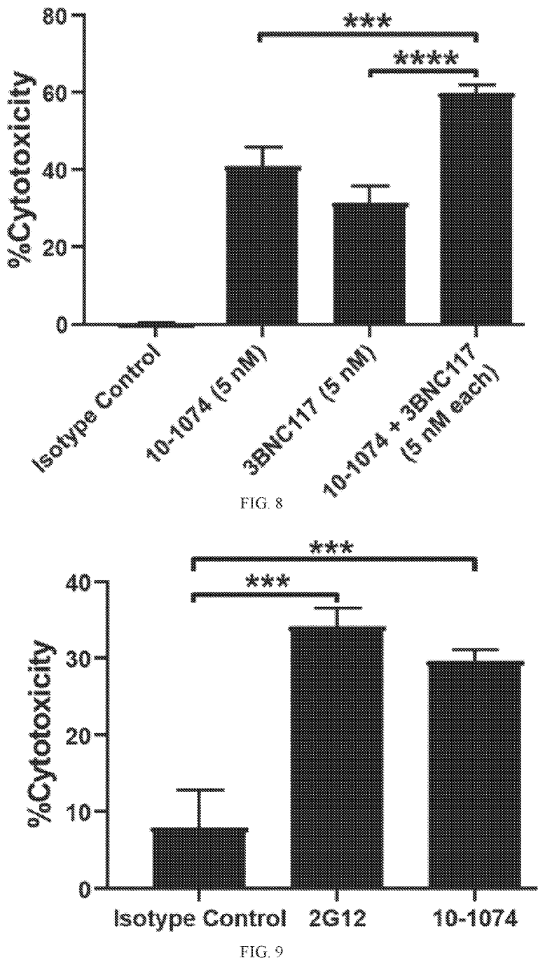

Finally, we examined whether the same technique could be used to expand the specificity of CAR-T cells. Primary human T cells were activated with anti-CD3/CD28 beads and recombinant IL-2 for 3 days and then transduced with the lentiviral vector encoding anti-DNP CAR. Flow cytometric analysis showed that about 36% of T cells expressed anti-DNP CARs. CAR-T cells were first blocked with an antihuman CD4 antibody (clone: RPA-T4) to prevent syncytia formation between CD4+ T cells and gp160+ cells and then cocultured with subtype B gp160⁺ HEK293 cells at a 1:1 E/T ratio in the presence of 10 nM of DNP-modified bNAbs or isotype control. After a 48-h incubation at 37° C., cell viability was assessed by flow cytometry. The result showed that DNP-conjugated 2G12 and 10-1074 enabled anti-DNP CAR-T cells to kill about 34% and 29% of gp160+ cells, respectively (FIG. 9). It is thus clear that the technique described here applies to both T cells and NK cells.

In this proof-of-concept study, we developed a new approach that can potentially enable CAR-NK cells, as well as CAR-T cells, to target and eradicate multiple variants of HIV-infected cells. Our strategy involves generating an anti-DNP CAR-NK cell that can be redirected to target various epitopes of HIV-1 gp160 using DNP-conjugated antibodies as adaptor molecules. We showed that the universal CAR-NK cells, as well as universal CAR-T cells, could be redirected to recognize and kill both subtypes B and C gp160⁺ cell lines and HIV-infected human primary T cells. Given that numerous anti-gp160 antibodies with different specificities are readily available, this approach can significantly expand the epitope coverage of anti-HIV CAR-NK cells, thus providing a promising strategy to overcome viral diversity.

Importantly, we find that bNAbs targeting membrane-distal regions of gp160 (including V1/V2, V3, and CD4bs) have a much higher likelihood of activating universal CAR-NK cells against gp160⁺ cells than those targeting membrane-proximal epitopes located in the gp41 MPER. Our results are in contrast to previous studies on anticancer CAR-T cells and bispecific T cell engagers (BiTE), which report that targeting membrane-proximal epitopes confers more effective T-cell activation than targeting membrane-distal epitopes. For example, CAR-T cells have been actively developed to target different regions of CD22, a B-cell leukemia antigen containing seven extracellular immunoglobulin (Ig)-like domains (numbered 1-7, from distal to proximal). James et al. reported that targeting the most distal Ig domains 1 and 2 (by the HD39-derived CAR) was less effective than targeting Ig domain 3 (by the RFB4-derived CAR) in J. Immunol. (2008) 180, 7028-7038, and Haso et al. further found that the latter was less effective than targeting the most proximal Ig domains 5-7 (by the m971-derived CAR) in Blood (2013) 121, 1165-1174.

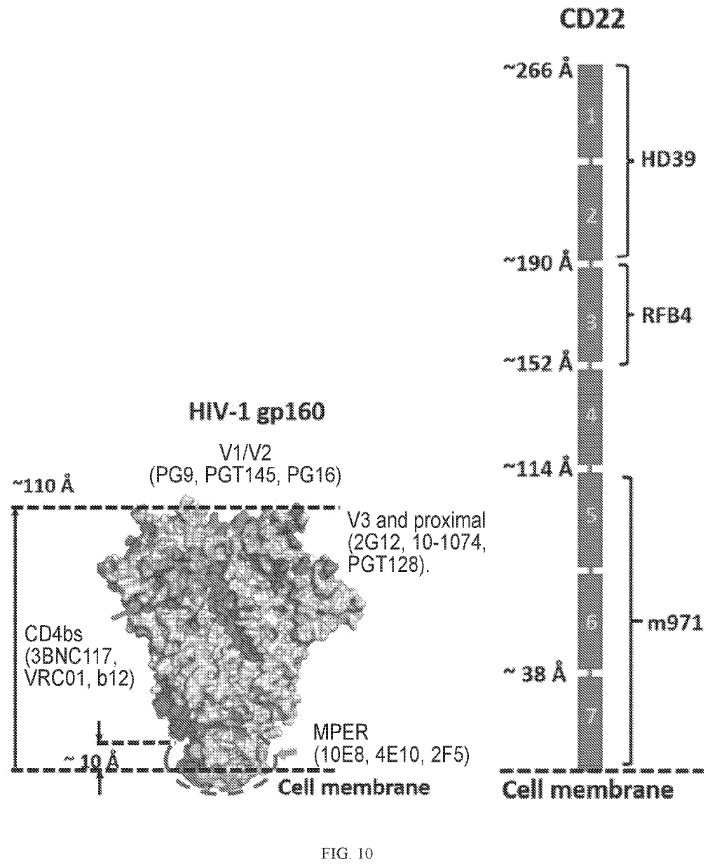

The discrepancy between our findings and others may stem from the poor accessibility of membrane-proximal epitopes of gp160. These epitopes, located in the HIV-1 gp41 MPER, are incredibly close to the cell membrane, e.g., within 10 Å for the 10E8 epitope (FIG. 10). As such, they may become hardly accessible due to local steric hindrance, consistent with our observation that there is little or no staining of gp160⁺ cells by 10E8 (FIGS. 3D and 4C). The membrane-proximal epitopes in previous cancer studies are not nearly as proximal as HIV-1 MPER epitopes. For example, the m971 and RFB4 epitopes of CD22 are up to 114 Å and 152-190 Å from the cell membrane, respectively. They are similar to or even farther away than the membrane-distal epitopes of HIV-1 gp160, which are within 110 Å from the cell membrane (FIG. 10). There is thus a significant variation in actual membrane proximity among different membrane-proximal epitopes, which may explain the contradictory results from our study and previous studies. Taken together, we speculate that there is an optimal distance between the epitope and the cell membrane to activate CAR-T or CAR-NK cells. If the epitope is too membrane-distal, it will allow the protein phosphatase CD45, which has a large extracellular domain, to diffuse into the immunological synapse to inhibit lymphocyte signaling, as indicated by a kinetic segregation model. However, if the epitope is too membrane-proximal, its accessibility will deteriorate, countering the positive effects of CD45 exclusion on lymphocyte activation. The optimal epitope distance may also be adjustable by using CARs with different hinge lengths.

Another possible cause of the discrepancy is that many HIV-1 MPER-targeting bNAbs preferably bind to the fusion-intermediate state over the prefusion conformation of gp41. In this study, we expect that gp160 is in the prefusion state because it lacks binding to its receptor CD4 and coreceptor CCR5 or CXCR4. This factor may have contributed to the reduced binding efficacy between MPER-targeting bNAbs and gp160⁺ cells, which in turn weakens the cytotoxicity of CAR-NK cells.

This study is the first to engineer NK cells with a universal CAR and to apply it for targeting HIV-1 epitopes. It is important to note that allogeneic T cells from healthy donors carry a high risk of inducing GVHD due to the expression of highly diverse T cell receptors, while allogeneic NK cells have little or no such risk. Therefore, the universal CAR-NK cell can potentially be developed as an off-the-shelf cellular therapeutic for all patients. We also conceive creating allogeneic T cells that do not induce GVHD, e.g., by disabling T cell receptors, in a universal anti-DNP CAR T cell approach.

Our universal CAR-NK cell approach has several important potential advantages. First, the universal CAR is more versatile because it is compatible with all types of antibodies, including IgA which cannot effectively induce NK cell-mediated ADCC. Second, it is likely more specific and safer, because anti-DNP CAR-NK cell-mediated killing is strictly dependent on DNP-conjugated antibodies and thus will not be induced by serum antibodies. Third, the potency of universal CAR-NK cells can be further enhanced by incorporating multiple signaling domains. Indeed, it was recently reported that CARs containing the trans-membrane and intracellular signaling domains of NKG2D, 2B4, DAP10, and CD3ζ could allow NK cells to more effectively kill ovarian cancer cells, as compared to a CAR containing the CD16 intracellular signaling domain. Finally, the universal CAR-NK cell approach and ADCC are not mutually exclusive because the DNP-conjugated bNAbs can still recruit primary NK cells to respond to gp160⁺ cells.

Although up to 1% of naturally-generated antibodies in humans can recognize DNP, which can compete with the DNP-conjugated adaptor molecules in binding and stimulating anti-DNP CAR-NK cells, we conceive that high affinity anti-DNP CARs overcomes this issue, as the recognition of DNP-conjugated adaptor molecules is strengthened with these universal CAR-NK cells.

For continued enhancement of the potency of the universal CAR-NK cells, it is conceived to use NK cell-specific signaling domains, such as NKG2D, 2B4, and CD16, which have led to enhanced anti-cancer CAR-NK cells. It is also conceived to incorporate DNP into anti-gp160 antibodies at an optimal site. It is also conceived to validate the universal CAR-NK cells using human primary CD4⁺ T cells infected with various HIV-1 strains and humanized mouse models of HIV-1 infection.

In summary, our proof-of-concept study demonstrates that a single universal CAR-NK cell can be redirected to target various epitopes of HIV-1 envelope glycoprotein gp160. Given that numerous anti-gp160 antibodies are readily available, this modular approach can significantly expand the epitope coverage of CAR-NK cells, making it possible to overcome the extraordinary diversity and mutability of HIV-1. An attractive potential of our universal CAR-NK cell platform is that it can potentially be developed as a low-cost, off-the-shelf cellular therapeutic for eradicating HIV infection in many patients.

### Methods of Preparation

Construction of the anti-DNP CAR lentiviral vector. The human CD8 α signal peptide was used to translocate the anti-DNP CAR to the cellular membrane. The gene fragment consisting of the CD8 α signal peptide, HA-tag, and anti-DNP scFv was synthesized by Integrated DNA Technologies (IDT, San Diego, CA). It was then amplified by PCR using primers CD8 signal-BamHI-F and αDNP-R. The gene fragment consisting of the CD8 α chain hinge domain, the CD28 transmembrane domain, the CD28 intracellular domain, and the CD3ζ intracellular domain was amplified from a parental lentiviral plasmid pFUW-anti-CD19-CD28-CD3ζ encoding an anti-CD19 CAR (a gift from Prof. Pin Wang at USC), using primers CD8 hinge-F and CD28-CD3ζ-EcoR I-R. The two gene fragments were linked together by overlap PCR, digested with BamH I and EcoR I, and ligated into the pFUW linear vector. The ligation product was transformed into DH5α cells. Single colonies grown on LB-agar plates containing 100 μg/ml ampicillin were picked and grown in the LB liquid medium. The plasmid was purified, sequence verified, and then used to generate lentiviral particles by calcium-phosphate transfection of HEK293T cells.

Generation of anti-DNP CAR-NK cells. To generate the lentivirus, HEK293T cells (˜600,000 cells/mL) were plated in a 100 mm dish and transfected the next day with 13 μg of the pFUW-aDNP CAR and 6 μg of lentiviral packaging plasmids (pVSVG, pRRE, and pREV) using Lipofectamine 2000 reagent. After 4 hours of incubation at 37° C., the supernatant was changed to fresh complete DMEM media with 10% FBS and antibiotics and incubated at 37° C. for lentivirus production. After three days of incubation, the lentivirus supernatant was filtered using a 0.45 μm Supor membrane filter and concentrated ˜10X using an Amicron 100 kDa MWCO concentrator. Anti-DNP CAR-NK cells were generated by lentiviral transduction using the retronectin-based technique. Non-treated 24-well plates were coated with 10 μg/mL retronectin at room temperature for 2 hours. Plates were then blocked using a 1X phosphate buffered saline (PBS) supplemented with 2% bovine serum albumin (BSA) solution at room temperature for 30 minutes. After blocking, the plate was washed two times using sterile 1X PBS before lentiviral transduction. To initiate the transduction, about ˜400,000 cells/mL of NK-92MI cells in RPMI complete culture medium supplemented with 20% FBS were plated per well and mixed with an equal volume of CAR lentivirus in the retronectin-coated plate. Then the plate was centrifuged at 1,200 g for 90 minutes followed by overnight incubation at 37° C. The next day, the transduced NK-92MI cells were washed to remove the excess lentivirus and cultured in RPMI complete culture medium with 20% FBS. After 48 hours, the transduction efficiency was verified by flow cytometry using the LSRII (BD Biosciences, San Jose, CA). CAR-NK cells were enriched two times using the MagniSort streptavidin positive selection beads.

Generation of gp160-expressing cell lines. HEK293 cells were transfected with the plasmid pConBgp160-opt (for subtype B gp160) or pConCgp160-opt (for subtype C gp160) (45 μg/each) and Lipofectamine 2000 (144 μL) in a 15 cm cell culture dish. After incubation at 37° C. for 6 hours, the excess Lipofectamine and plasmid were removed by media changes. After an additional two-day incubation, G418 (400 μg/mL for Subtype B, 500 μg/mL for Subtype C) was added to the cell culture to select for cells expressing these genes. All cell colonies after selection were collected. A small aliquot was stained with VRC01 and PE-conjugated secondary anti-human IgG polyclonal antibodies and analyzed by flow cytometry to confirm gp160 expression. The rest of the cells were further purified by FACS using a BD FACSAria II sorter. After further growth, cells were stained and analyzed by flow cytometry using the LSRII flow cytometer (BD Biosciences, San Jose, CA) to further verify the purity and the expression efficiency of gp160.

IFN-γ production assays. The gp160-expressing HEK293 cells were incubated with DNP-conjugated bNAb or isotype control at the specified concentration(s) for 20 minutes at room temperature. Next, antibody-treated gp160-expressing cells and DNP-CAR NK cells were co-cultured at a 1:1 E:T ratio in a U-bottom 96-well plate (50,000 cells each per well, in triplicate). Cells were incubated in an incubator maintained at 37oC and with 5% CO2. After 4 hours of incubation, cells were centrifuged. Supernatant from each well (100 μL) was collected, and the concentration of IFN-γ was analyzed using an ELISA kit (Thermo Fisher Scientific) by following the manufacturer's instructions.

Flow cytometry-based cytotoxicity assays. The subtype B or C gp160-expressing HEK293 cells were labeled with carboxyfluorescein succinimidyl ester (2.5 μM) in 1X PBS (supplemented with 2% FBS) for 5 min at room temperature, and washed with RPMI 1640 complete medium (supplemented with 10% FBS) for three times. The carboxyfluorescein succinimidyl ester-reacted, labeled gp160-expressing cells and the non-labeled gp160-negative cells (50,000/each) were mixed at a 1:1 ratio and then incubated with one of the seven DNP-conjugated antibodies (VRC01, 3BNC117, 2G12, 10-1074, PG9, 10E8, and isotype control) at a concentration of 2 nM for 20 min at room temperature. Then anti-DNP CAR-NK cells were added to the mixture of gp160-positive and negative cells at either 1:1, 5:1, or 25:1 E:T ratios (with respect to DNP-CAR NK cells and gp160⁺ cells) in triplicate. After 8 hours of incubation at 37° C., cells were collected and stained with APC-labeled human anti-HLA-A2 antibody for separating HEK293 cells (HLA-A2⁺) from CAR-NK cells (HLA-A2⁻) and aqua live/dead cell stain reagent. Cells were analyzed by flow cytometry using the LSRII (BD Biosciences, San Jose, CA), and the cytotoxic activity was assessed based on the number of remaining live gp160⁺ cells, calculated using the FlowJo software (Ashland, OR).

Cytotoxicity Assays against HIV-Infected Primary CD4+ T Cells. Primary CD4+ T cells were purified from healthy PBMCs with CD4 microbeads (Miltenyi #130-045-101) and stimulated with plate-bound anti-CD3 and soluble anti-CD28 antibodies for 2 days. Then, cells were infected with HIV-1 NL4-3 (500 ng of p24 per million cells) and cultured in 5 ng/mL IL-2 (Peprotech human IL-2 #200-02-1MG) for 10 days. Infected CD4 cells were prestained with Celltrace Farred (ThermoFisher #C34564), incubated with or without DNP-conjugated antibodies (10 nM) for 20 min, and then cocultured with anti-DNP CAR-NK cells at a 20:1 E/T ratio for 16 h. For the negative control, infected CD4 T cells were cultured without anti-DNP CAR-NK cells. After 16 h of coincubation, cells were stained with zombie violet fixable viability dye (Biolegend #423113), fixed, and stained intracellularly using a BD Cytofix/Cytoperm kit (BD #554714) for HIV core antigen (Clone KC57, Beckman Coulter, #6604667). Cells were analyzed by flow cytometry using a MACSQuant analyzer 10 (Miltenyi) and FlowJo (BD).

Generation of anti-DNP CAR-T Cells. Cryopreserved human PBMCs (10×10⁶ cells) were thawed, washed, and resuspended in 8 mL of complete RPMI 1640 medium supplemented with 10% FBS and then stimulated with 50 IU/mL of recombinant IL-2 and 75 μL of Human T-Activator CD3/CD28 Dynabeads (ThermoFisher #11131D) for 3 days. The stimulated PBMCs (1×10⁶ cells) were transferred to a nontreated 24-well plate precoated with retronectin (10 μg/mL). Anti-DNP CAR lentiviral particles were generated by calcium phosphate transfection of HEK293T cells and were subsequently concentrated using Lenti-X Concentrator solution (Takara Biosciences #631232). Then the lentivirus supernatant was added to PBMCs, followed by spinoculation with 100 IU/mL IL-2 at 1200 g for 90 min at RT. After transduction, PBMCs were washed three times and then cultured for a week in complete RPMI medium supplemented with 50 IU/mL IL-2 and Human T-Activator Dynabeads. CAR expression was verified by flow cytometry, and CAR-T cells were used directly in the cytotoxicity assay against gp160+ cells

$$( 1 - \frac{gp 160 + cellsremainingforeachbNAb}{gp 160 + cellsremaininginthenoantibodycontrol} ) \times 100$$

Safety Statement. Lentivirus vectors were used in this study to engineer NK cells with chimeric antigen receptors. These vectors are classified as level 2 biohazard material.

Construction of the traditional 2G12 CAR lentiviral vector pFUW-2G12-CD28-CD3ζ. The variable regions of anti-HIV-1 gp120 monoclonal antibody 2G12 were used to construct 2G12 scFv [VH-(GGGGS)₃ (SEQ ID NO:1)-VL]. An additional GGGGS (SEQ ID NO:2)-based linker, i.e., SEQ ID NO:1, was incorporated in between HA-tag and 2G12 scFv. The gene fragment consisting of the CD8 α signal peptide, HA-tag, GGGGS (SEQ ID NO:2)-based linker, and 2G12 scFv was synthesized by Integrated DNA Technologies (IDT, San Diego, CA). It was then amplified by PCR using primers CD8 signal-BamH I-F and 2G12-R. This fragment was then linked together with CD8 hinge-CD28-CD3z by overlap PCR and digested with BamH I and EcoR I. It was then ligated into the pFUW linear vector, and the ligation product was transformed into DH5α cells. Single colonies grown on LB-agar plates containing 100 μg/ml ampicillin were picked and grown in the LB liquid medium. The plasmid was purified, sequence verified, and then used to generate lentiviral particles by calcium-phosphate transfection of HEK293T cells.

Conjugation of PE to DNP. R-PE (1 mg/mL, Columbia Biosciences) was reacted with DNP-NHS ester at a molar ratio of 1:20 in 0.1 M sodium bicarbonate buffer, pH 8.3. After incubation at room temperature for 4 hours, the excess DNP-NHS was removed, and the buffer was changed into 1X PBS with 0.5% sodium azide utilizing a 40 kDa MWCO 0.5 mL Zeba Spin Column. The number of DNP moieties per R-PE molecule was determined based on measuring the absorbance at 280 nm (for R-PE) and 360 nm (for DNP) using the Nanodrop 2000 spectrophotometer (Thermo Fisher Scientific, Waltham, MA). The average number of DNP moieties per R-PE was calculated using the following equation:

$$[ DNP ] / [ R - PE ] =$$

$$( A 360 / ( ε\_molar DNP ( 17 , 300 M \hat{} ( - 1 ) cm \hat{} ( - 1 ) ) ) ) / ( ( A\_ 280 -$$

$$( A\_ 360 \times 0.31 ) ) / ( ε\_molar R -$$

$$PE ( 1 , 960 , 000 M \hat{} ( - 1 ) cm \hat{} ( - 1 ) ) ) ) \frac{[ DNP ]}{[ R - PE ]} =$$

$$\frac{\frac{{A}_{360}}{{ε}_{molar} DNP ( 17 , 300 {M}^{- 1} {cm}^{- 1} )}}{\frac{{A}_{280} - ( {A}_{360} \times 0.31 )}{{ε}_{molar} R - PE ( 1 , 960 , 000 {M}^{- 1} {cm}^{- 1} )}} .$$

Conjugation of bNAbs to DNP. All antibodies, including PG9, PGT145, PG16, 2G12, PGT128, 10-1074, VRC01, b12, 3BNC117, 10E8, 4E10, 2F5, and human IgG (1 mg/mL), were conjugated to DNP using the DNP-NHS ester (molar ratio 1:5) in 0.1M sodium bicarbonate buffer, pH 8.3. After overnight incubation at 4° C., the excess DNP-NHS reagent was removed, and the buffer was changed into 1X PBS buffer containing 0.5% sodium azide, using a 40 kDa MWCO 0.5 mL Zeba Spin Column. The average number of DNP moieties per antibody was determined by measuring the absorbance at 280 nm (for antibodies) and 360 nm (for DNP) using a Nanodrop 2000 spectrophotometer (Thermo Fisher Scientific, Waltham, MA). The final number of DNP moieties per antibody was calculated using the following equation:

$$( [ DNP ] ) / ( [ Antibody ] ) =$$

$$( A 360 / ( ε\_molar DNP ( 17 , 300 M \hat{} ( - 1 ) cm \hat{} ( - 1 ) ) ) ) / ( ( A\_ 280 -$$

$$( A\_ 360 \times 0.31 ) ) / ( ε\_molar Antibody ( 210 , 000 M \hat{} ( - 1 ) cm \hat{} ( - 1 ) ) ) ) .$$

$$\frac{[ DNP ]}{[ Antibody ]} = \frac{\frac{{A}_{360}}{{ε}_{molar} DNP ( 17 , 300 {M}^{- 1} {cm}^{- 1} )}}{\frac{{A}_{280} - ( {A}_{360} \times 0.31 )}{{ε}_{molar} Antibody ( 210 , 000 {M}^{- 1} {cm}^{- 1} )}} .$$

Verification of DNP Conjugation to 2G12. DNP-conjugated and unconjugated antibodies 2G12 (2 μg/each) in 1X sample loading buffers were loaded onto pre-made 8-16% Tris-Glycine gels. After SDS-PAGE of two identical gels, the first gel was stained with GelCode Blue Stain Coomassie reagent to confirm that both reagents were loaded in equal amounts, and the second gel was subjected to western blotting analysis. To verify DNP conjugation by western blotting, proteins were first transferred to a PVDF membrane using the Transblot Turbo Blotting System. Then the membrane was blocked for 1 hour using 5% milk solution and incubated with a 1:500 dilution of the anti-DNP primary antibody overnight at 4° C. The membrane was washed three times using 1X Tris Buffered Saline with 0.05% Tween20 (1X TBST), incubated with a 1:5000 dilution of the donkey anti-goat IgG HRP secondary antibody for one hour at room temperature, and then washed three times using 1X TBST. Next, the membrane was incubated with an enhanced chemiluminescent (ECL) substrate and then imaged using the ChemiDoc Gel Imaging System (BioRad, Hercules, CA).

Flow cytometry-based antibody binding assay. The subtype B or C gp160-expressing HEK293 cells were stained with either VRC01, 3BNC117, 2G12, 10-1074, PG9, 10E8, or the isotype control at a 1:3 serial dilution from 300 nM to 0.045 nM. In the negative control, no antibody was added. To detect antibody binding, each sample was additionally stained using a PE-conjugated anti-human IgG secondary antibody (4 μg/mL). Stained cells were analyzed by flow cytometry using the LSRII (BD Biosciences, San Jose, CA), and the average fluorescence intensity per cell was analyzed using the FlowJo software (Ashland, OR).

Statistical analysis. All cell culture experiments were performed in triplicate, and data were presented as Mean ±SD. For verification of anti-DNP CAR-NK cell activation against subtype B gp160-expressing cells with DNP-conjugated 2G12, two-way ANOVA was conducted using the 2G12 antibody, gp160-negative and positive cells as the independent variables. For assessing the optimal 2G12 adaptor molecule concentration for universal anti-DNP-CAR activation, one-way ANOVA was conducted using the 2G12 antibody as the independent variable. For validation of anti-DNP CAR-NK cell activation against subtype B and subtype C gp160-expressing cells by IFN-γ production, one-way ANOVA was conducted using adaptor molecule types as the independent variable. For comparison of the different bNAbs in directing anti-DNP-CAR cytotoxic activity against subtype B and subtype C gp160-expressing cells, a two-way ANOVA was conducted using antibody types and different E:T ratios as the independent variables. For comparison of the universal to the conventional 2G12 CAR-NK cell by flow cytometry-based cytotoxicity assays, a two-way ANOVA was conducted using CAR-NK cell types and different E:T ratios as the independent variables. For the comparison of the DNP-conjugated and unconjugated PG9 antibody in activating anti-DNP CAR-NK cells to produce IFN-γ, one-way ANOVA was conducted using the PG9 antibody type as the independent variable. For comparison of 10-1074 and 3BNC117 (either alone or in combination) in activating of universal CAR-NK cells, a one-way ANOVA was conducted using antibody type as the independent variable. For verification of anti-DNP CAR-T cell activation against subtype B gp160+ cells, a one-way ANOVA was conducted using antibody type as the independent variable. Both one-way and two-way ANOVA analyses were followed by either Tukey's or Dunnett's post hoc test for multiple comparisons. Significance was set at α=0.05. To draw the saturation curves for the bNAb binding assay to subtype B and C gp160-positive cells, a nonlinear regression curve fit, using the [Agonist] vs. response (three parameters) setting, was conducted. All statistical analyses were carried out using GraphPad Prism 8 (San Diego, CA).

### Reagents

1. Anti-gp160 bNAbs
The following reagents were obtained through the NIH AIDS Reagent Program, Division of AIDS, NIAID, NIH: 1) Anti-HIV-1 gp120 Monoclonal (PG9) from IAVI; 2) Anti-HIV-1 gp120 Monoclonal (PGT145) from IAVI; 3) Anti-HIV-1 gp120 Monoclonal (PG16) from IAVI; 4) Anti-HIV-1 gp120 Monoclonal (2G12) from Polymun Scientific; 5) Anti-HIV-1 gp120 Monoclonal (PGT128) from IAVI; 6) 10-1074 MAb from Dr. Michel C. Nussenzweig; 7) Anti-HIV-1 gp120 monoclonal (VRC01) from Dr. John Mascola (cat#12033); 8) Anti-HIV-1 gp120 Monoclonal (IgG1 b12) from Dr. Dennis Burton and Carlos Barbas; 9) Anti-HIV-1 gp120 Monoclonal (3BNC117) from Dr. Michel C. Nussenzweig; 10) HIV-1 anti-gp41 mAb (10E8) from Dr. Mark Connors; 11) Anti-HIV-1 gp41 Monoclonal (4E10) from Polymun Scientific; 12) Anti-HIV-1 gp41 Monoclonal (2F5) from Polymun Scientific (cat#1475);
2. HIV gp160-expressing plasmids
The following reagents were obtained through the NIH AIDS Reagent Program, Division of AIDS, NIAID, NIH: 1) pConBgp160-opt (Cat#11402) from Dr. Beatrice Hahn; and 2) pConCgp160-opt (Cat#11407) from Dr. Beatrice Hahn.
3. Lentivirus plasmids
The parental pFUW vector encoding an anti-CD19 CAR as well as the other three plasmids (pVSVG, pREV, and pRRE) for the third generation lentivirus system were kind gifts from Prof. Pin Wang (University of Southern California).
4. PCR primers

CD8 signal-BamH I-F:
(SEQ ID NO: 3)
5′-ACGTGGATCCGCCACCATGGCTC-3′
αDNP-R:
(SEQ ID NO: 4)
5′-GGGTCTGGGTGCTGGAGTTGTAGTTGCAGAGACAGTGACCAGAGTCC
C-3′
CD8 hinge-F:
(SEQ ID NO: 5)
5′- ACTACAACTCCAGCACCCAGACCC -3′
CD28-CD3ζ-EcoR I-R:
(SEQ ID NO: 6)
5′-AGTCGAATTCTCATCATCTTGGTGGCAGAG-3′
2G12-R:
(SEQ ID NO: 7)
5′-GGGTCTGGGTGCTGGAGTTGTAGTCCTCTTGATCTCCACCCTGGTGC-
3′

5. Other reagents
HA-tag polyclonal rabbit antibody, F(ab′)2-donkey anti-rabbit IgG (H+L) secondary antibody PE, Goat anti-human IgG Fc secondary antibody PE, donkey anti-goat IgG (H+L) secondary antibody HRP, human IgG isotype control (Catalog No. 12000C), human anti-HLA-A2-APC (clone BB7.2), and human anti-CD56 (NCAM) APC were purchased from Thermo Fisher Scientific. The anti-human CD19 antibody (clone FMC63) was purchased from Novus Biologicals. Goat anti-dinitrophenol polyclonal antibody was purchased from Eagle Biosciences. CellTrace carboxyfluorescein succinimidyl ester (CFSE) cell proliferation kit, CellTrace Blue Stain Reagent kit, LIVE/DEAD fixable aqua dead cell stain kit, Lipofectamine 2000, Geneticin (G418) solution, Magnisort streptavidin positive selection beads, Human T-Activator CD3/CD28 Dynabeads, recombinant human interleukin-2 (IL-2) (Catalog No. 34-8029-85), 8-16% Tris-Glycine SDS-PAGE gel, Gel Code Blue Stain Reagent, 2X sample loading buffer, 40 kDa MWCO 0.5 mL Zeba Spin Column, and IFN-γ human uncoated ELISA kit were purchased from Thermo Fisher Scientific. Transblot Turbo Mini PVDF Transfer Packs, Transblot Turbo System, and Clarity Western ECL Substrate were from BioRad. Retronectin and Lenti-X Concentrator were purchased from Takara Biosciences. N-(2,4-Dinitrophenyl)-6-aminocaproic acid N-succinimidyl ester (DNP-NHS ester) and β-mercaptoethanol were purchased from Sigma Aldrich. Amicron 100 kDa MWCO concentrator was purchased from Millipore. RPMI, DMEM, fetal bovine serum, non-essential amino acids, sodium pyruvate, penicillin-streptomycin-glutamine were purchased from Thermo Fisher Scientific. Ficoll-Paque Plus Reagent was purchased from GE Healthcare.
6. Cell culture
The NK-92MI cell line was purchased from ATCC. The HEK293T cell line was a gift from Prof. Pin Wang (USC). Human buffy coats were purchased from Zen-Bio Inc (Research Triangle Park, NC). Human peripheral blood mononuclear cells (PBMC) were isolated from the buffy coat using Ficoll-Paque density gradient centrifugation. The HEK293 cell line was a gift of Prof. Wei-Chiang Shen (USC). NK-92MI cells were cultured in RPMI media supplemented with 20% fetal bovine serum and 0.1 mM non-essential amino acids, 1 mM sodium pyruvate, 0.5 mg/mL penicillin-streptomycin-glutamine, and 50 μM β-mercaptoethanol. HEK293T cells were cultured in the DMEM medium supplemented with 10% fetal bovine serum and 0.5 mg/mL of penicillin-streptomycin-glutamine (PSG). HEK293 cells were also cultured in DMEM medium supplemented with 10% FBS, but without PSG in order to enable for selection with Geneticin. Human PBMCs were cultured in RPMI media supplemented with 10% fetal bovine serum and 0.1 mM non-essential amino acids, 1 mM sodium pyruvate, 0.5 mg/mL penicillin-streptomycin-glytamine, and 50 μM β-mercaptoethanol. PBMCs were activated using Human T-Activator CD3/CD28 Dynabeads and 50 IU/mL recombinant IL-2 cytokine for 3 days before lentivirus transduction with anti-DNP CAR. Then primary human T-cells were further cultured using 50 IU/mL in complete RPMI medium before functional assays.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A combination for use as a treatment, comprising:
a genetically engineered immune cell expressing a chimeric antigen receptor (CAR) specific for 2,4-dinitrophenyl (DNP), and
at least one DNP-modified antibody, wherein the DNP-modified antibody is an antibody having a binding affinity specific for a marker associated with human immunodeficiency virus (HIV) or malignant B cells and modified with a DNP moiety,
wherein the genetically engineered immune cell expressing the CAR specific for the DNP recognizes the at least one DNP-modified antibody, so as to target a cell expressing the marker to which the DNP-modified antibody has the binding affinity.

2. A combination, comprising:
a polynucleotide encoding a chimeric antigen receptor (CAR) specific for 2,4-dinitrophenyl (DNP), or a vector comprising said polynucleotide; and
at least one DNP-modified antibody, wherein the DNP-modified antibody has a binding affinity specific for a marker associated with human immunodeficiency virus (HIV) or malignant B cells,
wherein the polynucleotide or the vector upon introduction into an immune cell results in a genetically engineered immune cell expressing the CAR specific for DNP, said genetically engineered immune cell recognizes the at least one DNP-modified antibody and thereby target a cell expressing the marker to which the DNP-modified antibody has the binding affinity.

3. The combination of claim 2, further comprising a quantity of immune cells.

4. The combination of claim 1, wherein the immune cell comprises a NK cell, or a T cell.

5. (canceled)

6. The combination of claim 1,
wherein the DNP-modified antibody has a binding affinity specific for an HIV-1 envelop glycoprotein gp160, gp 120, or gp41, or
wherein the DNP-modified antibody has a binding affinity specific for B-lymphocyte antigen CD19, or
wherein the DNP-modified antibody modified has at least two moieties of the DNP per molecule of the antibody.

7. The combination of claim 6, wherein the DNP-modified antibody is a broadly neutralizing HIV-1 antibody (bNAb) selected from a group consisting of PG9, PGT145, PG16, 2G12, PGT128, PGT121, 10-1074, VRC01, b12, 3BNC117, 3BNC60, 10E8, 4E10, and 2F5, said bNAb is modified with a DNP moiety.

8. The combination of claim 7, wherein the at least one DNP-modified antibody modified comprises two or more bNAbs selected from the group, said two or more bNAbs are each modified with a DNP moiety.

9. (canceled)

10. (canceled)

11. The combination of claim 1, wherein the CAR specific for DNP comprises:
a. a DNP-specific targeting region, comprising a variable light chain (V_{L}) of an anti-DNP antibody, a variable heavy chain (V_{H}) of an anti-DNP antibody, or both,
b. a transmembrane domain, and
c. an intracellular signaling domain.

12. The combination of claim 11, wherein the DNP-specific targeting region is a single-chain variable fragment (scFv) comprising the V_{L} having a polypeptide sequence of SEQ ID NO: 11, 15, or 19, the V_{H} having a polypeptide sequence of SEQ ID NO: 12, 16, or 20, respectively, and a peptide linker positioned between the V_{L} and the V_{H}.

13. A chimeric antigen receptor (CAR) specific for 2,4-dinitrophenyl (DNP), comprising:
a. a DNP-specific targeting region, comprising a variable light chain (V_{L}) of an anti-DNP antibody, a variable heavy chain (V_{H}) of an anti-DNP antibody, or both,
b. a transmembrane domain, and
c. an intracellular signaling domain.

14. The CAR of claim 13, wherein the transmembrane domain comprises CD28TM, and the intracellular signaling domain comprises one or more of NKG2D, 2B4, DAP10, CD28, CD16, and CD3ζ.

15. The CAR of claim 13, wherein the DNP-specific targeting region comprises:
the V_{L} having a polypeptide sequence of SEQ ID NO: 11, 15, or 19;
the V_{H} having a polypeptide sequence of SEQ ID NO: 12, 16, or 20;
a combination comprising the V_{L} having a polypeptide sequence of SEQ ID NO: 11 and the V_{H} having a polypeptide sequence of SEQ ID NO: 12;
a combination comprising the V_{L} having a polypeptide sequence of SEQ ID NO: 15 and the V_{H} having a polypeptide sequence of SEQ ID NO: 16; or
a combination comprising the V_{L} having a polypeptide sequence of SEQ ID NO: 19 and the V_{H} having a polypeptide sequence of SEQ ID NO: 20.

16. A genetically engineered immune cell expressing the chimeric antigen receptor (CAR) specific for 2,4-dinitrophenyl (DNP) of claim 13.

17. The genetically engineered immune cell of claim 16, comprising a natural killer (NK) cell, or a T cell.

18. (canceled)

19. A polynucleotide encoding the chimeric antigen receptor (CAR) specific for 2,4-dinitrophenyl (DNP) of claim 13.

20. A vector, comprising a polynucleotide of claim 19.

21. A method for inducing apoptosis of HIV-1-infected cells or malignant B cells, comprising:
contacting the HIV-1-infected cells or the malignant B cells with the genetically engineered immune cell and the at least one DNP-modified antibody of the combination of claim 1.

22. A method for inducing apoptosis of HIV-1-infected cells or malignant B cells, comprising:
inducing expression of a CAR specific for DNP in an immune cell by introducing the polynucleotide or the vector in the combination of claim 2 into the immune cell, thereby generating a genetically engineered immune cell expressing the CAR specific for DNP, and
contacting the HIV-1-infected cells or the malignant B cells with the genetically engineered immune cell in the presence of the at least one DNP-modified antibody in the combination of claim 2.

23. (canceled)

24. A method for inducing apoptosis of HIV-1-infected cells or malignant B cells, comprising:
contacting a genetically engineered immune cell according to claim 16 with the HIV-1-infected cells or the malignant B cells in the presence of a DNP-modified antibody, wherein the DNP-modified antibody has a binding affinity specific for a marker associated with the HIV-1 or the malignant B cells and is modified with a DNP moiety.

25. The method of claim 21, further comprising measuring an amount of IFN-γ in a medium culturing the genetically engineered immune cell during and/or after the contact, and/or measuring a level of cytotoxicity of the HIV-infected cells and/or the malignant B cells during and/or after the contact.

26. The method of claim 21, wherein the genetically engineered cell is provided in a plurality and contacted with the HIV-infected cells or the malignant B cells at a number ratio ranging from 25:1 to 1:1.

27. The method of claim 21, wherein the DNP-modified antibody is present in a concentration of between 2 nM and 50 nM.

28. The method of claim 21, for inducing apoptosis of HIV-1-infected cells, said HIV-1-infected cells expressing subtype B or subtype C envelop protein, comprising:
detecting expression of subtype B envelop protein in the HIV-1-infected cells, and contacting the genetically engineered immune cell with the HIV-1-infected cells detected with the expression of subtype B envelop protein in the presence of one or a cocktail of DNP-modified, HIV-specific bNAbs selected from PG9, PGT145, PG16, 2G12, PGT128, 10-1074, and 3BNC117, said bNAbs are each modified with a DNP moiety; or
detecting expression of subtype C envelop protein in the HIV-1-infected cells, and contacting the genetically engineered immune cell with the HIV-1-infected cells detected with the expression of subtype C envelop protein in the presence of one or a cocktail of DNP-modified, HIV-specific bNAbs selected from PG9, 10-1074, 3BNC117, and VRC01, said bNAbs are each modified with a DNP group.

29. A method of treating a subject suffering from or inflicted with HIV, comprising:
administering to the subject an effective amount of the genetically engineered immune cell and the at least one DNP-modified antibody of the combination of claim 1, so as to reduce or eradicate the number of HIV-infected cells in the subject.

30. A method of treating a subject suffering from or inflicted with HIV, comprising:
inducing expression of a CAR specific for DNP in an immune cell by introducing the polynucleotide or the vector of the combination of claim 2 into the immune cell, thereby generating a genetically engineered immune cell expressing the CAR specific for DNP; and
administering the genetically engineered immune cell and administering the at least one DNP-modified antibody of the combination of claim 2 to the subject, so as to reduce or eradicate the number of HIV-infected cells in the subject.

31. (canceled)

32. A method of treating a subject suffering from or inflicted with HIV, comprising:
administering to the subject an effective amount of a genetically engineered immune cell of claim 16 for reducing or eradicating the number of HIV-infected cells in the subject.

33. The method of claim 32, further comprising: administering to the subject an effective amount of two or more DNP-modified antibodies, wherein the DNP-modified antibodies are HIV-specific bNAbs, each modified with a DNP moiety.

34. The method of claim 29,
wherein HIV-infected cells of the subject express subtype B envelop protein gp160, gp120 or gp41, and the at least one HIV-specific bNAb comprises PG9, PGT145, PG16, 2G12, PGT128, 10-1074, 3BNC117, or a combination thereof, said at least one HIV-specific bNAb is modified with a DNP moiety, or
wherein HIV-infected cells of the subject express subtype C envelop protein gp160, gp120 or gp41, and the at least one HIV-specific bNAb comprises PG9, 10-1074, 3BNC117, VRC01, or a combination thereof, said at least one HIV-specific bNAb is modified with a DNP moiety, or
wherein HIV-infected cells of the subject express both subtype B and subtype C envelop protein gp160, gp120 or gp41, and the at least one HIV-specific bNAb comprises at least one selected from PG9, PGT145, PG16, 2G12, PGT128, 10-1074, and 3BNC117, each modified with a DNP moiety, and at least one selected from PG9, 10-1074, 3BNC117, and VRC01, each modified with a DNP moiety.

35. (canceled)

36. (canceled)
